SEQUENCE LISTING (19 sequences)

SEQ ID NO: 1 - DNA, 143 bp, Artificial Sequence
  Synthetic
ttacccctagtgatggagttgcccactccctctctgcgcgctcgctcgctcggtggggcctgcggaccaaaggtccgcagacggcagagctctgctctgccggccccaccgagcgagcgagcgcgcagagagggagtgggcaa

SEQ ID NO: 2 - DNA, 145 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaa

SEQ ID NO: 3 - DNA, 146 bp, Artificial Sequence
  Synthetic
gccatacctctagtgatggagttggccactccctctatgcgcactcgctcgctcggtggggcctggcgaccaaaggtcgccagacggacgtgctttgcacgtccggccccaccgagcgagcgagtgcgcatagagggagtggccaa

SEQ ID NO: 4 - DNA, 146 bp, Artificial Sequence
  Synthetic
gggcaaacctagatgatggagttggccactccctctatgcgcgctcgctcactcactcggccctggagaccaaaggtctccagactgccggcctctggccggcagggccgagtgagtgagcgagcgcgcatagagggagtggccaa

SEQ ID NO: 5 - DNA, 143 bp, Artificial Sequence
  Synthetic
atacccctagtgatggagttgcccactccctctatgcgcgctcgctcgctcggtggggcctgcggaccaaaggtccgcagacggcagaggtctcctctgccggccccaccgagcgagcgagcgcgcatagagggagtgggcaa

SEQ ID NO: 6 - DNA, 145 bp, Artificial Sequence
  Synthetic
cggtacccctagtgagggagttggccactccctctatgcgcgctcgctcgctcggtggggcctgcggaccaaaggtccgcagacggcagagctctgctctgccggccccaccgagcgagcgagcgcgcatagagggagtggccaa

SEQ ID NO: 7 - DNA, 130 bp, Artificial Sequence
  Synthetic
aggaacccctagtgatggagttggccactccctctctgctcactcactcactcactgaggccagggcaccaaaggtgccctgacagccctgctttgcagggctggcctcagtgagtgagtgagtgagcag

SEQ ID NO: 8 - DNA, 128 bp, Artificial Sequence
  Synthetic
gcgcgctcgctcgctcactgaggccgcccgggcaaagcccgggcgtcgggcgacctttggtcgcccggcctcagtgagcgagcgagcgcgcagagagggagtggccaactccatcactaggggttcct

SEQ ID NO: 9 - DNA, 128 bp, Artificial Sequence
  Synthetic
gctcactcactcactcactgaggccagccctgcaaagcagggctgtcaggccacctttggtggcctggcctcagtgagtgagtgagtgagcagagagggagtggccaactccatcactaggggttcct

SEQ ID NO: 10 - DNA, 130 bp, Artificial Sequence
  Synthetic
aggaacccctagtgatggagttggccactccctctctgcgcgctcgctcgctcactgaggccgggcgaccaaaggtcgcccgacgcccgggctttgcccgggcggcctcagtgagcgagcgagcgcgcag

SEQ ID NO: 11 - DNA, 131 bp, Artificial Sequence
  Synthetic
aggaacccctagtgatggagttggccactccctctctgctcactcactcactcactgaggcctggggaccaaaggtccccagactgcagagctttgctctgcaggcctcagtgagtgagtgagtgagcaga

SEQ ID NO: 12 - DNA, 128 bp, Artificial Sequence
  Synthetic
gctcactcactcactcactgaggcctgcagagcaaagctctgcagtctggggacctttggtccccaggcctcagtgagtgagtgagtgagcagagagggagtggccaactccatcactaggggttcct

SEQ ID NO: 13 - DNA, 24 bp, Artificial Sequence
  Synthetic
acataaggtcaatgggaggtaagc

SEQ ID NO: 14 - DNA, 23 bp, Artificial Sequence
  Synthetic
caatgggactttcctgttgattc

SEQ ID NO: 15 - DNA, 20 bp, Artificial Sequence
  Synthetic
gatgtgctgcaaggcgatta

SEQ ID NO: 16 - DNA, 20 bp, Artificial Sequence
  Synthetic
ttatgcttccggctcgtatg

SEQ ID NO: 17 - DNA, 20 bp, Artificial Sequence
  Synthetic
ggcttgggtaaactgggaaa

SEQ ID NO: 18 - DNA, 24 bp, Artificial Sequence
  Synthetic
gttcacagagactactgcacttat

SEQ ID NO: 19 - DNA, 24 bp, Artificial Sequence
  Synthetic
atgtggtgtactggctccaccttt

(19) United States
(12) Patent Application Publication
(10) Pub. No.: US 2023/0250422 A1
(43) Pub. Date: Aug. 10, 2023

(54) CPG-FREE ITRS FOR AAV GENE THERAPY

(71) Applicant: THE CURATORS OF THE UNIVERSITY OF MISSOURI, Columbia, MO (US)

(72) Inventors: DONGSHENG DUAN, Columbia, MO (US); XIUFANG PAN, Columbia, MO (US); YONGPING YUE, Columbia, MO (US)

(21) Appl. No.: 17/995,763

(22) PCT Filed: Apr. 7, 2021
(86) PCT No.: PCT/US2021/026262

§ 371 Date: Oct. 7, 2022

Related U.S. Application Data
(60) Provisional application No. 63/006,148, filed on Apr. 7, 2020.

(51) Int. Cl. C12N 15/11; C12N 15/86; A61P 21/00

(52) U.S. Cl. CPC C12N 15/11; C12N 15/86; A61P 21/00; C12N 2750/14143

(57) ABSTRACT

Disclosed are recombinant adeno-associated virus (rAAV) nucleic acid vectors comprising inverted terminal repeats (ITRs) free of 5′-cytosine-phosphate-guanine-3′ (CpG) motifs. Also disclosed are rAAV particles comprising the rAAV vectors, and to compositions and methods for delivering nucleic acids and/or for gene therapy. Further disclosed are compositions and methods for treating diseases with AAV gene therapy using the rAAV vector.

### CROSS-REFERENCE TO RELATED APPLICATIONS

This application claims the benefit to U.S. Provisional Application Ser. No. 63/006,148, filed Apr. 7, 2020, which is incorporated herein by reference in its entirety.

### STATEMENT REGARDING FEDERALLY SPONSORED RESEARCH OR DEVELOPMENT

This invention was made with Government support under grant numbers NS090634 and AR070517 awarded by the National Institutes of Health and grant number W81XWH-14-1-0302 awarded by the Army Medical Research and Material Command. The Government has certain rights in the invention.

### INCORPORATION OF SEQUENCE LISTING

A paper copy of the Sequence Listing and a computer readable form of the Sequence Listing containing the file named “20UMC037_ST25.txt”, which is 5,645 bytes in size (as measured in MICROSOFT WINDOWS® EXPLORER), are provided herein and are herein incorporated by reference. This Sequence Listing consists of SEQ ID NOs:1-19.

### BACKGROUND OF THE DISCLOSURE

The present disclosure relates generally to recombinant adeno-associated virus (rAAV) nucleic acid vectors comprising inverted terminal repeats (ITRs) free of 5′-cytosine-phosphate-guanine-3′ (CpG) motifs (i.e., the ITRs do not include any CpG motifs). More particularly, the present disclosure relates to rAAV particles comprising the rAAV vector, to compositions and methods for delivering nucleic acids, and to compositions and methods for gene therapy. The present disclosure further relates to compositions and methods for treating diseases with AAV gene therapy using the rAAV vector.

Adeno-associated virus (AAV) is a helper-dependent parvovirus first discovered as a contaminating particle in the adenovirus stock. AAV contains a ˜4.7 kb single-stranded DNA genome. AAV was developed in late 80s and early 90s as a gene delivery/gene therapy vector. Three AAV vectors have been approved by regulatory agencies for treating inherited diseases. These include Glybera for treating lipoprotein lipase deficiency, Luxturna (Voretigene neparvovec-rzyl) for treating Leber congenital amaurosis, and Zengensma (Onasemnogene abeparvovec-xioi) for treating spinal muscular atrophy. AAV gene therapy has also resulted in remarkable clinical success in many other genetic diseases such as hemophilia A, hemophilia B, X-linked myotubular myopathy, and giant axonal neuropathy.

A rAAV vector is generated by replacing the wild-type AAV replication (Rep) and structural/capsid (Cap) open reading frames with a transgene expression cassette. The two inverted terminal repeats (ITRs) are the only wild-type viral sequences in the rAAV vector (FIG. 1A). Each ITR consists of nucleotides that form a T-shaped hairpin structure in either a flip or a flop configuration.

The ITR is essential for both wild-type AAV and rAAV genome replication, progeny genome generation and encapsidation, and conversion of the single-stranded vector genome to the transcription competent latent form for persistent transgene expression. AAV vector production depends on the successful rescue of the vector genome from the double stranded proviral plasmid (cis-plasmid), the subsequent replication of the vector genome through a self-priming mechanism, and the displacement and encapsidation of the single stranded genome into a pre-assembled capsid. The ITR is essential for all these processes. Specifically, the large Rep proteins bind to the RBE and RBE′ elements. These interactions position the large Rep proteins to make a sequence- and strand-specific nick at the terminal resolution site (trs). The free 3′ OH group created by this cleavage serves as the replication primer for the synthesis of the secondary ITR. Further replication leads to the production of a new complementary strand and the displacement of the original complementary strand. The displaced strand (vector genome) is pumped through a 5-fold channel into a pre-formed empty capsid in a 3′ to 5′ direction by the small Rep proteins. In addition to the critical role played in vector production, the ITR is also important for AAV transduction. The ITR-primed single-strand to double-strand conversion of the vector genome is a prerequisite for the transcription of transgene. Persistent AAV transduction (persistent transgene expression) also relies inter-ITR recombination and subsequent formation of the episomal circular AAV genome.

Despite significant advance in translating AAV gene therapy from the bench to the bedside, there are still important hurdles. Among these is the immune response. AAV was initially considered a weakly immunogenic vector. However, recent animal study results and clinical trial data suggest that the AAV vector can induce a significant immune response via both the innate and adaptive immune mechanisms.

These shortcomings highlight the need to develop new, enhanced AAV gene therapy techniques. Accordingly, there exists a need to develop AAV gene therapy vectors with improved immunogenicity properties.

Genetic modifications of ITRs may represent an approach to reduce the immunogenicity of the AAV vector. Unfortunately, ITR mutagenesis has been notoriously associated functional deficiency.

The structure-function relationship of the ITR has been extensively interrogated by mutagenesis. Most ITR mutations are deleterious. They negatively impact AAV replication and/or encapsidation (Ryan et al., 1996; Wang et al., 1998; Brister and Muzyczka, 1999; 2000; McCarty et al., 2003; Zhong et al., 2008; Zhou et al., 2008; Ling et al., 2015; Zhou et al., 2017). Dinucleotide transversion mutation of the RBE reduces Rep binding by 2 to 10-fold (Ryan et al., 1996). Single nucleotide transversion mutation of the core sequence of the RBE results in up to 5-fold reduction in Rep binding (Ryan et al., 1996). Single nucleotide transversion mutation of the trs nearly abolishes ITR nicking by the large Rep proteins (Brister and Muzyczka, 1999). Truncation of the B and C arm leads to an 8-fold decrease in AAV replication (Zhou et al., 2017). Deletion of the trs in one ITR completely prevents AAV genome replication from the mutated ITR (McCarty et al., 2003; McCarty, 2008). Deletion and/or substitution of the D-sequence renders AAV to package only the plus or the minus strand genome, instead of both (Zhong et al., 2008; Zhou et al., 2008; Ling et al., 2015). Defective ITR has also been associated with the packaging of non-vector sequences (Wang et al., 1996; Wang et al., 1998; Savy et al., 2017; Tai et al., 2018). Collectively, these studies reveal the importance of maintaining an intact ITR in AAV gene therapy.

### BRIEF DESCRIPTION OF THE DISCLOSURE

The present disclosure relates generally to inverted terminal repeats (ITRs) free of 5′-cytosine-phosphate-guanine-3′ (CpG) motifs (i.e., the ITRs do not include any CpG motifs). More particularly, the present disclosure relates to recombinant adeno-associated virus (rAAV) nucleic acid vectors including ITRs free of CpG motifs (i.e., the ITRs do not include any CpG motifs). The present disclosure also relates to rAAV particles and pharmaceutical compositions comprising the rAAV vector. The present disclosure also relates to methods for delivering nucleic acids and to methods for AAV gene therapy. The present disclosure further relates to compositions and methods for treating diseases with AAV gene therapy using the rAAV vector wherein the ITRs of the rAAV vector are free of CpG motifs (i.e., the ITRs do not include any CpG motifs).

In one aspect, the present disclosure is directed to inverted terminal repeats (ITRs) free of 5′-cytosine-phosphate-guanine-3′ (CpG) motifs (i.e., the ITRs do not include any CpG motifs).

In one aspect, the present disclosure is directed to a recombinant adeno-associated virus (rAAV) nucleic acid vector comprising inverted terminal repeats (ITRs) free of 5′-cytosine-phosphate-guanine-3′ (CpG) motifs (i.e., the ITRs do not include any CpG motifs).

In another aspect, the present disclosure is directed to an rAAV particle comprising a viral capsid and a rAAV nucleic acid vector comprising ITRs free of 5′-cytosine-phosphate-guanine-3′ (CpG) motifs (i.e., the ITRs do not include any CpG motifs).

In another aspect, the present disclosure is directed to a pharmaceutical composition comprising a pharmaceutically acceptable carrier and an rAAV nucleic acid vector comprising ITRs free of (CpG) motifs. Preferably, the ITRs are CpG-free ITRs (i.e., the ITRs do not include any CpG motifs).

In another aspect, the present disclosure is directed to a method of delivering nucleic acids into a cell, the method comprising administering to the cell an rAAV vector comprising ITRs free of 5′-cytosine-phosphate-guanine-3′ (CpG) motifs.

In another aspect, the present disclosure is directed to a method of treating a disease in a subject in need thereof, the method comprising administering to the subject a therapeutically effective amount of an rAAV vector comprising ITRs free of 5′-cytosine-phosphate-guanine-3′ (CpG) motifs. Preferably, the ITRs are CpG-free ITRs (i.e., the ITRs do not include any CpG motifs).

### BRIEF DESCRIPTION OF THE DRAWINGS

The disclosure will be better understood, and features, aspects and advantages other than those set forth above will become apparent when consideration is given to the following detailed description thereof. Such detailed description makes reference to the following drawings, wherein:

FIG. 1A depicts a schematic outline of an AAV vector.

FIG. 1B depicts the alignment of the 3′-ITR from AAV1 (SEQ ID NO:1), 2 (SEQ ID NO:2), 3 (SEQ ID NO:3), 4 (SEQ ID NO:4), 6 (SEQ ID NO:5), 7 (SEQ ID NO:6) and the version-1 of the CpG-free AAV (AAV CpG-free 1; SEQ ID NO:7).

FIG. 1C depicts a two-dimensional drawing of the wild-type ITR (SEQ ID NO:8) and mutations made in the version-1 CpG-free ITR (SEQ ID NO:9) to eliminated CpG motifs at the 5′-end of the vector genome (5′-ITR) in the flop configuration.

FIG. 1D depicts a two-dimensional drawing of the 3′-ITR in the flop configuration showing the wild type 3′-ITR (130 nucleotide) sequence (SEQ ID NO:10) and base changes resulting in a CpG-free 3′-ITR (SEQ ID NO:7).

FIG. 1E depicts the alignment of the 3′-ITR from AAV1 (SEQ ID NO:1), 2 (SEQ ID NO:2), 3 (SEQ ID NO:3), 4 (SEQ ID NO:4), 6 (SEQ ID NO:5), 7 (SEQ ID NO:6) and the version-2 of the CpG-free AAV (AAV CpG-free 2; SEQ ID NO:11).

FIG. 2A depicts quantification of the vector yield from three independent production rounds for each vector.

FIG. 2B depicts representative transmission electron microscopy images of the wild-type ITR vector and CpG-free ITR vector.

FIG. 2C depicts quantification of empty particles.

FIG. 3A depicts representative dystrophin immunofluorescence staining and HE staining micrographs from the tibialis anterior muscle of dystrophin-null mdx mice that did not receive AAV micro-dystrophin injection (uninjected, right panel), injected with the CpG-free AAV micro-dystrophin vector (CpG-free ITR, left panels), and the wild-type AAV micro-dystrophin vector (wild-type ITR, middle panels).

FIG. 3B depicts quantification of dystrophin positive myofibers in the tibialis anterior muscle of dystrophin-null mdx mice that received either the CpG-free AAV micro-dystrophin vector (CpG-free ITR) or the wild-type AAV micro-dystrophin vector (wild-type ITR).

FIG. 3C depicts western blot evaluation of micro-dystrophin expression in the tibialis anterior muscle of dystrophin-null mdx mice that did not receive AAV micro-dystrophin vector injection (uninjected), injected with the CpG-free AAV micro-dystrophin vector (CpG-free ITR), and the wild-type AAV micro-dystrophin vector (wild-type ITR).

FIG. 3D depicts quantification of the dystrophin expression level by western blot in the tibialis anterior muscle of dystrophin-null mdx mice that received either the CpG-free AAV micro-dystrophin vector (CpG-free ITR) or the wild-type AAV micro-dystrophin vector (wild-type ITR).

FIG. 3E depicts quantification of the AAV vector genome copy number by quantitative PCR in the tibialis anterior muscle of dystrophin-null mdx mice that received either the CpG-free AAV micro-dystrophin vector (CpG-free ITR) or the wild-type AAV micro-dystrophin vector (wild-type ITR).

FIGS. 4A-4F depict representative full-view dystrophin immunostaining and hematoxylin and eosin (H&E) staining photomicrographs of the tibialis anterior muscle.

FIGS. 5A and 5B depict evaluations of centronucleation and myofiber size distribution.

FIGS. 6A-6H depict quantitative evaluations of muscle contractility.

While the disclosure is susceptible to various modifications and alternative forms, specific embodiments thereof have been shown by way of example in the drawings and are herein described below in detail. It should be understood, however, that the description of specific embodiments is not intended to limit the disclosure to cover all modifications, equivalents and alternatives falling within the spirit and scope of the disclosure as defined by the appended claims.

### DETAILED DESCRIPTION OF THE DISCLOSURE

Unless defined otherwise, all technical and scientific terms used herein have the same meaning as commonly understood by one of ordinary skill in the art to which the disclosure belongs. Although any methods and materials similar to or equivalent to those described herein can be used in the practice or testing of the present disclosure, the preferred methods and materials are described below.

The approach of the present disclosure is to produce ITRs that lack one or more CpG motifs relative to wild-type ITRs. Preferably, the ITRs are CpG-free ITRs. In some aspects, the ITRs are used in rAAV vectors. Surprisingly, the ITRs retain functionality for gene delivery despite the mutations. An important advantage of this approach is that the rAAV vectors do not include any CpG motifs (i.e., lack any CpG motifs; also referred to herein to be “CpG-free”).

In one aspect, the present disclosure is directed to inverted terminal repeats (ITRs) lacking at least one 5′-cytosine-phosphate-guanine-3′ (CpG) motif. Preferably, the ITRs do not include any CpG motifs (i.e., are “CpG-free”). Preferably, the ITR is one of SEQ ID NO:7, SEQ ID NO:9, SEQ ID NO:11, and SEQ ID NO:12.

In one aspect, the present disclosure is directed to a recombinant adeno-associated virus (rAAV) nucleic acid vector comprising inverted terminal repeats (ITRs) lacking one or more 5′-cytosine-phosphate-guanine-3′ (CpG) motifs. Preferably, the ITRs do not include any CpG motifs (i.e., are “CpG-free”). Preferably, the ITR is one of SEQ ID NO:7, SEQ ID NO:9, SEQ ID NO:11, and SEQ ID NO:12.

As used herein, 5′-cytosine-phosphate-guanine-3′ (CpG) motif refers to a cytosine (C) and a guanine (G) separated by one phosphate group in a single-stranded linear sequence. The CpG notation is used to distinguish this single-stranded linear sequence from the CG base-pairing of cytosine and guanine for double-stranded sequences. Without being bound by particular theory, it is believed that reducing the number of the CpG motifs of the ITR attenuates the T cell response and prolongs transgene expression. It is believed that, following uptake, unmethylated CpG motif is sensed by Toll-like receptor 9 (TLR9) in the endosome of plasmacytoid dendritic cells (Zhu et al., 2009; Martino et al., 2011; Toth et al., 2019), and that this leads to the production of type I interferon and activation of cytotoxic T lymphocyte cells (Zhu et al., 2009; Rogers et al., 2015; Rogers et al., 2017; Ashley et al., 2019).

As used herein, recombinant adeno-associated virus nucleic acid vector, or rAAV vector, refers to single-stranded deoxyribonucleic acid (ssDNA) chain that carries a 5′-ITR at the 5′-end of the genome and a 3′-ITR at the 3′-end of the genome. The DNA between the 5′-ITR and 3′-ITR can be an expression cassette that may be used to carry genetic material into a foreign cell. The term rAAV vector may refer to the sequence of bases in the nucleic acid chain (the primary structure) or to the three-dimensional folded ssDNA molecule (the tertiary structure).

In some embodiments, recombinant adeno-associated virus nucleic acid vector, or rAAV vector, may also refer to self-complementary vectors which have a terminal resolution site mutated ITR in the middle and two open-ended regular ITRs at the 5′-end and 3′-end of the genome. The folding back of the 5′ half of the genome and the 3′ half of the genome forms a complementary double-stranded deoxyribonucleic acid (dsDNA) and is used to carry genetic material into a foreign cell.

As used herein, “wild-type AAV ITR(s)” refers to one or both of a 5′-ITR and a 3′-ITR, which are terminal ssDNA segments in naturally occurring adeno-associated viruses and recombinant AAV vectors. Example naturally occurring adeno-associated viruses include AAV1, AAV2, AAV3, AAV4, AAV5, AAV6, AAV7, AAV8, AAV9, AAV10, AAV11, AAV12, AAV13. A particularly suitable wild-type AAV ITR for use as a reference point in preparing the CpG-free ITRs of the present disclosure is from adeno-associated virus serotype 2 (AAV2). The term wild-type AAV ITR may refer to the sequence of bases in the nucleic acid chain (the primary structure) or to an ITR segment in the three-dimensional folded ssDNA AAV vector molecule (the tertiary structure). Typical rAAV vectors are devoid of all native viral sequences except the sequences for the ITRs. Therefore, many vectors used in gene therapy or other rAAV applications employ vectors including wild-type AAV ITRs. It is therefore to be understood that the term wild-type AAV ITRs as used herein is inclusive of the wild-type AAV ITRs used in many rAAV vectors. Exemplary wild type AAV ITR sequences include SEQ ID NO:1, SEQ ID NO:2, SEQ ID NO:3, SEQ ID NO:4, SEQ ID NO:5, and SEQ ID NO:6.

Wild-type AAV ITRs contain about 145 nucleic acids. The ITR of the present disclosure can comprise about 100, about 110, about 120, about 130, about 140, about 150, about 160, about 170, about 180, about 190, about 200, about 100 to about 150, about 110 to about 150, about 110 to about 140, about 120 to about 140, about 120 to about 150, about 130 to about 150, or about 130 to about 140 nucleic acids. It was observed that deleting the terminal 15-17 nucleotides of the wild-type ITR (i.e. the 15-17 nucleotides of the 5′ end of the 5′-ITR and/or the 15-17 nucleotides of the 3′ end of the 3′-ITR) did not alter ITR function (Samulski et al., 1987; Savy et al., 2017), therefore particularly suitable ITRs of the present disclosure comprise about 130 nucleic acids.

In some embodiments, the ITR comprises about 70% to about 99%, about 70% to about 95%, about 70% to about 90%, about 70% to about 80%, about 80% to about 99%, about 80% to about 95%, or about 80% to about 90% sequence identity to one or more of SEQ ID NO:7, SEQ ID NO:9, SEQ ID NO:11, and SEQ ID NO:12. In some embodiments, the ITR comprises about 70%, about 75%, about 80%, about 85%, about 90%, about 91%, about 92%, about 93%, about 94%, about 95%, about 96%, about 97%, about 98%, or about 99% sequence identity to one or more of SEQ ID NO:7, SEQ ID NO:9, SEQ ID NO:11, and SEQ ID NO:12.

In some embodiments, the ITR lacks one or more CpG motifs contained in wild-type AAV ITRs. The two wild-type AAV ITRs in wild-type AAV vectors contain a total of 32 CpG motifs (16 in each). In some embodiments, an ITR lacks 1, 2, 3, 4, 5, 6, 7, 8, 9, 10, 11, 12, 13, 14, 15, or 16 CpG motifs in the wild-type AAV ITR. In some embodiments, the ITRs lack 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, or 32 CpG motifs in the wild-type AAV ITRs. In some embodiments, the ITR comprises at least 70%, at least 80%, at least 85%, at least 95%, or 99% sequence identity to one or more wild-type AAV ITRs, and the ITR lacks one or more of the 16 CpG motifs in the wild-type AAV ITR. Particularly suitable ITRs comprise at least 70%, at least 80%, at least 85%, at least 95%, or 99% sequence identity to one or more wild-type AAV ITRs of serotype-2 (AAV2 ITRs), and the ITR lacks one or more of the 16 CpG motifs in the wild-type AAV2 ITR. In preferred embodiments, the calculation of sequence identity disregards the terminal 15 nucleotides of the wild-type ITRs (i.e. the 15 nucleotides of the 5′ end of the 5′-ITR and/or the 15 nucleotides of the 3′ end of the 3′-ITR).

Preferably, the ITRs are free of CpG motifs (i.e., the ITR is a CpG-free ITR that does not include any CpG motifs.) As used herein, CpG-free ITR means the ITR does not include any CpG motifs. The two ITRs in wild-type AAV vectors contain a total of 32 CpG motifs (16 in each). In some embodiments, an ITR lacks the 16 CpG motifs in the wild-type AAV ITR. In some embodiments, the ITRs lack the 32 CpG motifs in the wild-type AAV ITRs. In some embodiments, the ITR comprises at least 70%, at least 80%, or at least 85% sequence identity to one or more wild-type AAV ITRs, and the ITR lacks the 16 CpG motifs in the wild-type AAV ITR. Particularly suitable ITRs comprise at least 70%, at least 80%, or at least 85% sequence identity to one or more wild-type AAV ITRs of serotype-2 (AAV2 ITRs), and the ITR lacks the 16 CpG motifs in the wild-type AAV2 ITR and/or the ITRs lack the 32 CpG motifs in the wild-type AAV2 ITRs. In preferred embodiments, the calculation of sequence identity disregards the terminal 15 nucleotides of the wild-type ITRs (i.e. the 15 nucleotides of the 5′ end of the 5′-ITR and/or the 15 nucleotides of the 3′ end of the 3′-ITR).

Percent identity of two sequences can be determined by aligning the sequences for optimal comparison. For example, gaps can be introduced in the sequence of a first nucleic acid sequence for optimal alignment with the second nucleic acid sequence. The nucleotides at corresponding positions are then compared. When a position in the first sequence is occupied by the same nucleotide as at the corresponding position in the second sequence, the nucleic acids are identical at that position. The percent identity between the two sequences is a function of the number of identical nucleotides shared by the sequences. Hence, percent identity=[number of identical nucleotides/total number of overlapping positions]×100. The percentage of sequence identity can be calculated according to this formula by comparing two optimally aligned sequences being compared, determining the number of positions at which the identical nucleic acid occurs in both sequences to yield the number of matched positions (the “number of identical positions” in the formula above), dividing the number of matched positions by the total number of positions being compared (the “total number of overlapping positions” in the formula above), and multiplying the result by 100 to yield the percent sequence identity. In this comparison, the sequences can be the same length or may be different in length. Optimal alignment of sequences for determining a comparison window can be conducted by the local homology algorithm of Smith and Waterman (1981) (Smith and Waterman, 1981), by the homology alignment algorithm of Needleman and Wunsh (1972) (Needleman and Wunsch, 1970), by the search for similarity via the method of Pearson and Lipman (1988) (Pearson and Lipman, 1988), by computerized implementations of these algorithms (GAP, BESTFIT, FASTA and TFASTA in the Wisconsin Genetics Software Package Release 7.0, Genetic Computer Group, 575, Science Drive, Madison, Wis.), or by inspection.

Preferably, the ITR lacks one or more of the 16 CpG motifs in the wild-type AAV ITRs due to point mutations of C or G residues in the CpG motifs. In particular embodiments, some or all of the point mutations of C or G residues in the CpG motifs are transition mutations. For example, the ITRs of the present disclosure can comprise about 85% sequence identity to wild-type ITRs (disregarding the deleted terminal 15 nucleotides of the wild-type ITRs in the sequence identity calculation), and can lack all 16 CpG motifs in the wild-type ITR with mutations of the ITR all being point mutations replacing cytosine [C] and/or guanine [G] in the wild-type ITR with adenine [A], or thymine [T], or guanine [G], or cytosine [C].

“Transition mutation” is used in accordance with its ordinary meaning as would be understood by a person of ordinary skill in the art, and occurs when a pyrimidine base (i.e., thymine [T] or cytosine [C]) substitutes for another pyrimidine base or when a purine base (i.e., adenine [A] or guanine [G]) substitutes for another purine base.

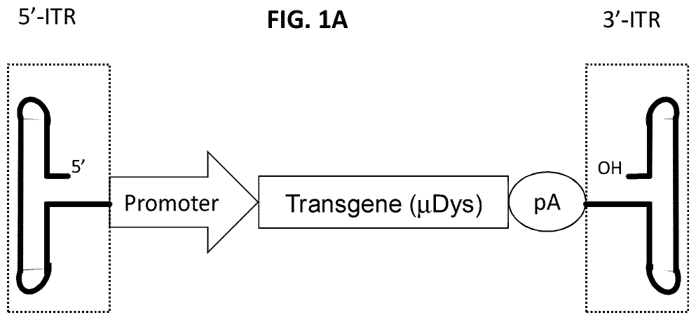

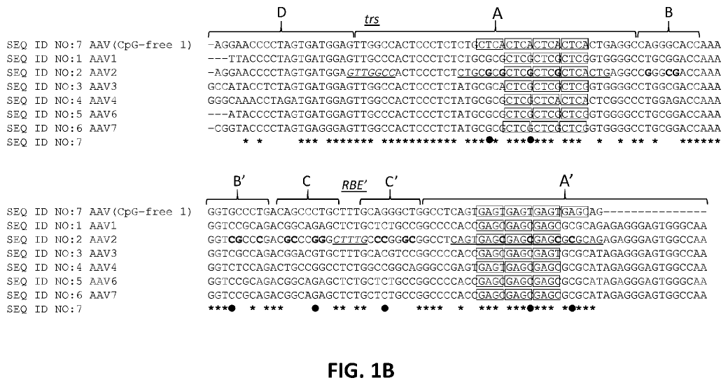

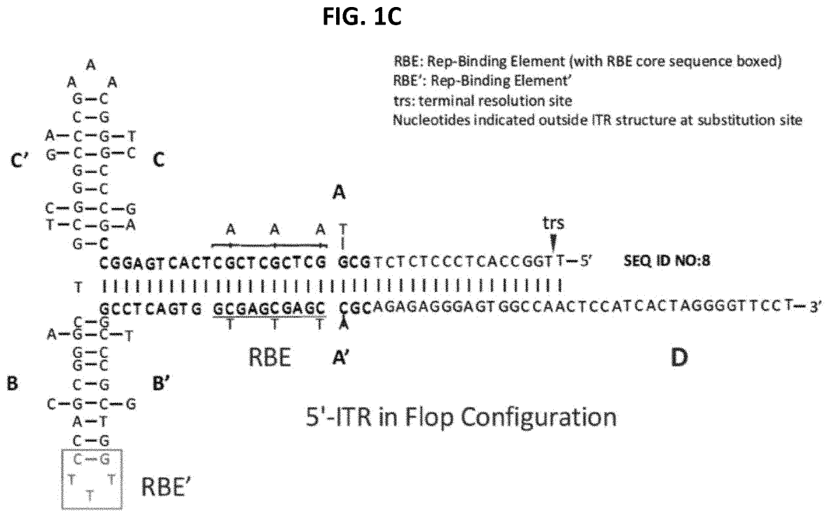

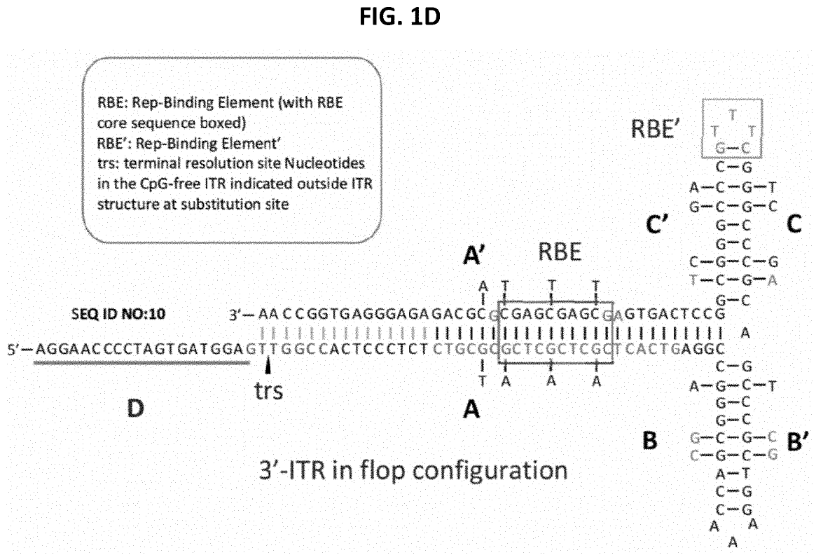

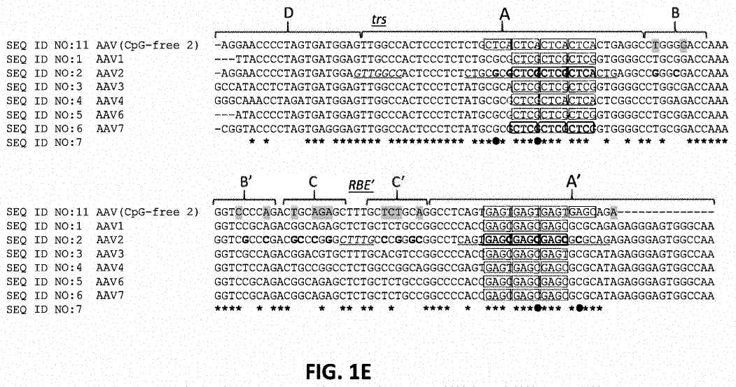

Wild-type ITRs can be divided into seven segments including the A, A′, B, B′, C, C′ and D sequence as in the exemplary 5′-ITR of AAV2 (FIG. 1C, SEQ ID NO:8). Sequence A, B and C are inversely complementary to sequence A′, B′ and C′, respectively. The pairing of sequences B/B′ and C/C′ forms the two arms of the T-shaped hairpin structure of the ITR. The pairing of sequence A and A′ forms the stem of the T-shaped ITR. The 20 nucleotide-long D sequence is maintained as the single stranded DNA in an AAV vector (FIGS. 1C and 1D).

Wild-type ITRs contain three sequence elements that are essential for function. These include the Rep binding element (RBE), the second Rep binding element (RBE′) and the terminal resolution site (trs). The RBE is located in the A/A′ stem and consists of a 22-bp sequence (FIGS. 1C and 1D). Within the RBE, there is a 10-bp core sequence (FIGS. 1C and 1D). Dinucleotide transversion mutations in the core sequence reduces the Rep binding affinity by at least 10-fold (Ryan et al., 1996). The three tetranucleotide repeats GAGY (RCTC in the complementary strand) is considered the consensus Rep-binding motif in the RBE (Amiss et al., 2003; Wilmott et al., 2019). Y refers to C or T and R reference to A or G. This consensus Rep-binding motif and its peripheral sequences are important for Rep binding (Wilmott et al., 2019). In some embodiments, the four tetranucleotide repeats GMGY (RCKC in the complementary strand) and its flanking sequences are considered important for Rep binding. M refers to A or C and K refers to G or T. In the context of AAV2 ITR, the four tetranucleotide repeats GMGC (GCKC in the complementary strand) and its flanking sequences (CAGT at the 5′-end and AG at the 3′-end) are required for Rep binding (Ryan et al., 1996). The RBE′ is located at the tip of either the B or the C arm. It consists of a 5-nucleotide sequence (FIGS. 1C and 1D) (Brister and Muzyczka, 2000). The trs is a 7-nucleotide sequence located at the junction of the A/A′ stem and the D-sequence (Brister and Muzyczka, 1999).

There are 16 CpG motifs in a wild-type ITR (FIGS. 1B and 1C). Mutations in these regions are known to affect ITR function (Ryan et al., 1996; Brister and Muzyczka, 1999; 2000; Zhou et al., 2017). These CpG motifs are located in the A/A′ stem (4 in sequence A, 4 in sequence A′), B/B′ arm (2 in sequence B, 2 in sequence B′) and C/C′ arm (2 in sequence C, 2 in sequence C′). Of three ITR essential elements, only the RBE contains the CpG motif (6 in the core sequence and 8 total). There is no CpG motif in the RBE′ and trs.

In some embodiments, the ITR of the present disclosure comprises a Rep binding element (RBE) comprising transition mutations. Preferably, all the mutations in the RBE are transition mutations. In some embodiments, the mutations in the ITR can include transition mutations, transversion mutations, and combinations thereof.

AAV vectors (wild-type and engineered) comprise two ITRs at either end of the vector, a 5′ end ITR and a 3′ end ITR (FIG. 1A). Accordingly, the rAAV vector of the present disclosure also comprises two ITRs, a 5′ end ITR and a 3′ end ITR, and at least one but preferably both ITRs lack one or more CpG motifs. More preferably, at least one but preferably both ITRs are CpG-free. In various embodiments the rAAV vector comprises a CpG-free 5′-end ITR, a CpG-free 3′-end ITR, and combinations thereof.

In some embodiments, the 5′-end ITR comprises a guanine to thymine substitution in a first CpG motif in an A segment of the 5′-end ITR. In some embodiments, 5′-end ITR comprises a guanine to adenine substitution in three remaining CpG motifs in the A segment.

In some embodiments, the 5′-end ITR comprises a guanine to adenine substitution in a first CpG motif in a C segment of the 5′-end ITR and a cytosine to guanine substitution in an immediate downstream cytosine in the C segment. In some embodiments, the 5′-end ITR comprises a guanine to cytosine substitution in a second CpG motif of a C segment of the 5′-end ITR and a guanine to thymine substitution in an immediate downstream guanine in the C segment.

In some embodiments, the 5′-end ITR comprises a guanine to thymine substitution in a first CpG motif in a C segment of the 5′-end ITR and a cytosine to guanine substitution in an immediate downstream cytosine in the C segment. In some embodiments, the 5′-end ITR comprises a cytosine to adenine substitution in a second CpG motif of a C segment of the 5′-end ITR and a guanine to adenine substitution in a guanine immediate downstream of the second CpG motif in the C segment.

In some embodiments, the 5′-end ITR comprises a guanine to adenine substitution in a first CpG motif in a B segment of the 5′-end ITR. In some embodiments, the 5′-end ITR comprises a guanine to cytosine substitution in a second CpG motif in a B segment of the 5′-end ITR.

In some embodiments, the 5′-end ITR comprises a guanine to thymine substitution in a first CpG motif in a B segment of the 5′-end ITR. In some embodiments, the 5′-end ITR comprises a cytosine to guanine substitution in a second CpG motif in a B segment of the 5′-end ITR.

In some embodiments, corresponding bases in A′, B′ and C′ segments of the 5′-end ITR are substituted with complementary bases.

In some embodiments, the 3′-end ITR comprises a guanine to thymine substitution in a first CpG motif in an A segment of the 3′-end ITR. In some embodiments, the 3′-end ITR comprises a guanine to adenine substitution in three remaining CpG motifs in the A segment of the 3′-end ITR.

In some embodiments, the 3′-end ITR comprises a guanine to adenine substitution in a first CpG motif in a B segment of the 3′-end ITR. In some embodiments, the 3′-end ITR comprises a cytosine to guanine substitution and a guanine to cytosine substitution in a second CpG motif of a B segment of the 3′-end ITR.

In some embodiments, the 3′-end ITR comprises a guanine to thymine substitution in a first CpG motif in a B segment of the 3′-end ITR. In some embodiments, the 3′-end ITR comprises a cytosine to guanine substitution in a second CpG motif of a B segment of the 3′-end ITR.

In some embodiments, the 3′-end ITR sequence comprises a guanine to adenine substitution in a first CpG motif in a C segment of the 3′-end ITR and a cytosine to guanine substitution in an immediate downstream cytosine in the C segment. In some embodiments, the 3′-end ITR sequence comprises a guanine to cytosine substitution in a second CpG motif in a C segment of the 3′-end ITR and a guanine to thymine substitution in an immediate downstream guanine in the C segment.

In some embodiments, the 3′-end ITR sequence comprises a guanine to thymine substitution in a first CpG motif in a C segment of the 3′-end ITR and a cytosine to guanine substitution in an immediate downstream cytosine in the C segment. In some embodiments, the 3′-end ITR sequence comprises a cytosine to adenine substitution in a second CpG motif in a C segment of the 3′-end ITR and a guanine to adenine substitution in an immediate guanine downstream of the second CpG motif in the C segment.

In some embodiments, corresponding bases in the A′, B′ and C′ segments of the 3′-end ITR are substituted with complementary bases.

The GC content is another important consideration in the design of the CpG-free ITR. The GC content of the 3′-ITR of wild-type AAV1, 2, 3, 4, 6, and 7 is 68.53%, 69.66%, 65.07%, 64.38%, 67.13%, and 68.97%, respectively. The GC content of the human genome is 40.9% on average. Particularly suitable CpG-free ITRs comprise a GC content less than 70%, less than 65%, less than 60%, of about 70%, of about 65%, of about 60%, ranging from about 40% to about 70%, ranging from about 40% to about 65%, or ranging from about 40% to about 60%. For example, in some embodiments the ITRs comprise a GC content of about 60%. In some embodiments, the ITRs comprise a GC content of 60.16% (5′-ITR of version-1 CpG-free ITR), 60.00% (3′-ITR of version-1 CpG-free ITR), 58.59% (5′-ITR of version-2 CpG-free ITR), and 58.02% (3′-ITR of version-2 CpG-free ITR).

The GC content is calculated with an online CC Content Calculator. Specifically, the GC-content percentage is calculated using the formula: count of total (G+C)/count of total (A+T+G+C)×100%.

Sequences for particularly suitable ITRs are presented in Table 1. In some embodiments, the ITRs comprise about 70%, about 75%, about 80%, about 85%, about 90%, about 95%, about 99%, or about 100% sequence identity to one or more of SEQ ID NO:7, SEQ ID NO:9, SEQ ID NO:11, and SEQ ID NO:12. Preferably, the ITRs comprise sequences selected from the group consisting of SEQ ID NO:7, SEQ ID NO:9, SEQ ID NO:11, and SEQ ID NO:12, and combinations thereof. For example, in one embodiment the 5′-end ITR comprises SEQ ID NO:9 and the 3′-end ITR comprises SEQ ID NO:7. In another embodiment the 5′-end ITR comprises SEQ ID NO:9 and the 3′-end ITR comprises SEQ ID NO:11. In another embodiment the 5′-end ITR comprises SEQ ID NO:12 and the 3′-end ITR comprises SEQ ID NO:11. In another embodiment the 5′-end ITR comprises SEQ ID NO:12 and the 3′-end ITR comprises SEQ ID NO:7.

TABLE 1
Sequence identification numbers (SEQ ID NO)

| SEQ | | |
|---|---|---|
| ID | | |
| NO | Name | Sequence |
| 9 | CpG-free | 5′- |
| | 5′-ITR | GCTCACTCACTCACTCACTGAGGCCAGCCCTGCAAAG |
| | (ver- | CAGGGCTGTCAGGCCACCTTTGGTGGCCTGGCCTCAGT |
| | sion-1) | GAGTGAGTGAGTGAGCAGAGAGGGAGTGGCCAACTC |
| | | CATCACTAGGGGTTCCT-3′ |
| 7 | CpG-free | 5′- |
| | 3′-ITR | AGGAACCCCTAGTGATGGAGTTGGCCACTCCCTCTCTG |
| | (ver- | CTCACTCACTCACTCACTGAGGCCAGGGCACCAAAGG |
| | sion-1) | TGCCCTGACAGCCCTGCTTTGCAGGGCTGGCCTCAGTG |
| | | AGTGAGTGAGTGAGCAG-3′ |
| 12 | CpG-free | 5′- |
| | 5′-ITR | GCTCACTCACTCACTCACTGAGGCCTGCAGAGCAAAG |
| | (ver- | CTCTGCAGTCTGGGGACCTTTGGTCCCCAGGCCTCAGT |
| | sion-2) | GAGTGAGTGAGTGAGCAGAGAGGGAGTGGCCAACTC |
| | | CATCACTAGGGGTTCCT-3′ |
| 11 | CpG-free | 5′- |
| | 3′-ITR | AGGAACCCCTAGTGATGGAGTTGGCCACTCCCTCTCTG |
| | (ver- | CTCACTCACTCACTCACTGAGGCCTGGGGACCAAAGG |
| | sion-2) | TCCCCAGACTGCAGAGCTTTGCTCTGCAGGCCTCAGTG |
| | | AGTGAGTGAGTGAGCAGA-3′ |

The rAAV vectors of the present disclosure further comprises an expression cassette. The expression cassette encodes for nucleic acid sequences of interest for delivery by AAV gene therapy techniques. In various embodiments, the expression cassette encodes for one or more diagnostic, therapeutic, and/or prophylactic agents. For example, in one embodiment the expression cassette encodes for micro-dystrophin.

The expression cassette can further encode a eukaryotic promoter. Particularly suitable eukaryotic promoters include tissue-specific promoters. In some embodiments, the expression cassette further encodes a tissue-specific promoter.

The expression cassette can further encode an inducible promoter. Suitable inducible promoters include, for example, a tetracycline (Tet)-inducible promoter, a doxycycline (Dox)-inducible promoter, and a tamoxifen (tam)-inducible promoter. Including an inducible promoter allows for temporal control over gene expression by administration of the inducing compound. For example, two components of the Tet- (and Dox-) inducible system are the Tet repressor (TetR) and the tet operator (tetO). Both Tet and its analog doxycycline (Dox) interact with TetR and are well tolerated and widely used in mammalian systems. The Tet-ON approach can be used to regulate gene expression. In the reverse Tet controlled transactivator (rtTA) or Tet-OFF system, Tet or Dox binds to and induces a Tet-responsive promoter.

### Delivery Systems

A suitable rAAV vector delivery method is delivery of naked DNA.

Preferably, the rAAV vector is included in a suitable DNA delivery system. Suitable DNA delivery systems include non-viral delivery systems. Particularly suitable non-viral delivery systems include, for example, liposomal vectors, cationic polymers, nanoparticles, and DNA binding polymers. In embodiments wherein more than one type of the rAAV vector is administered, the rAAV vectors can optionally be included in different delivery systems. Alternatively, in some embodiments, multiple rAAV vectors can be included in a single delivery system.

Another particularly suitable DNA delivery system includes viral capsids. Particularly suitable viral vectors include, for example, adenovirus, adeno-associated virus, lentivirus, retrovirus, Highlands J virus (HJV), human immunodeficiency virus (HIV), and Herpes simplex viruses (HSV). In embodiments wherein more than one type of rAAV vector is administered, the rAAV vectors can optionally be included in different viral capsids. Alternatively, in some embodiments, multiple rAAV vectors can be included in a single viral capsid.

An aspect of the present disclosure is directed to a recombinant adeno-associated virus (rAAV) particle comprising a viral capsid and a rAAV nucleic acid vector comprising inverted terminal repeats (ITRs) free of 5′-cytosine-phosphate-guanine-3′ (CpG) motifs.

A particularly suitable viral capsid is an AAV or an rAAV viral capsid. Some embodiments of the viral capsid are AAV1, 2, 3, 4, 5, 6, 7, 8, 9, 10, 11, 12, 13, rh10, rh74, and AAV-Anc80, AAV-B1, AAV-DJ, AAV-KP1, AAV-LK03, AAV-Myo, AAV-NP22, AAV-NP40, AAV-NP66, AAV-PHP.A, AAV-PHP.B, AAV tyrosine mutants or other naturally existing or laboratory generated capsids.

An AAV viral capsid refers to a wild-type viral capsid coded for by a wild-type AAV genome. The wild-type AAV genome includes a cap open reading frame that contains overlapping nucleotide sequences for capsid proteins VP1, VP2 and VP3, which interact to form a capsid with icosahedral symmetry. The molecular weights of these proteins are 87, 72 and 62 kiloDaltons, respectively. The wild-type AAV capsid is composed of a mixture of VP1, VP2, and VP3 totaling 60 monomers arranged in icosahedral symmetry in a ratio of 1:1:10, with an estimated size of 3.9 MegaDaltons. The rAAV nucleic acid vector may be encapsidated in the wild-type AAV capsid. In some embodiments, the viral capsid may be a modified version of a wild-type AAV capsid. For example, the rAAV nucleic acid vector may be encapsidated in a mutant AAV capsid or a recombinant AAV (rAAV) capsid.

The rAAV vector may be encapsidated in preassembled viral capsids by known methods.

### Pharmaceutical Compositions

Further aspects of the present disclosure are directed to pharmaceutical compositions including the rAAV vector or the rAAV particle described herein.

An aspect of the present disclosure is directed to a pharmaceutical composition comprising a pharmaceutically acceptable carrier and an rAAV nucleic acid vector comprising inverted terminal repeats (ITRs) free of 5′-cytosine-phosphate-guanine-3′ (CpG) motifs.

Another aspect of the present disclosure is directed to a pharmaceutical composition comprising a pharmaceutically acceptable carrier and a recombinant adeno-associated virus (rAAV) particle comprising a viral capsid and an rAAV nucleic acid vector comprising inverted terminal repeats (ITRs) free of 5′-cytosine-phosphate-guanine-3′ (CpG) motifs.

The compounds described herein can be formulated in a pharmaceutical composition by any conventional manner using one or more pharmaceutically acceptable carriers or excipients as described in, for example, Remington's Pharmaceutical Sciences (A. R. Gennaro, Ed.), 21st edition, ISBN: 0781746736 (2005), incorporated herein by reference in its entirety. Such compositions can contain a therapeutically effective amount (e.g., therapeutically effective amount) of one or more compounds described herein, which can be in purified form, together with a suitable amount of carrier so as to provide the form for proper administration to the subject.

The term “composition” refers to preparing a drug in a form suitable for administration to a subject, such as a human. Thus, a “composition” can include pharmaceutically acceptable excipients, including diluents or carriers.

The term “pharmaceutically acceptable” as used herein can describe substances or components that do not cause unacceptable losses of pharmacological activity or unacceptable adverse side effects. Examples of pharmaceutically acceptable ingredients can be those having monographs in United States Pharmacopeia (USP 29) and National Formulary (NF 24), United States Pharmacopeial Convention, Inc, Rockville, Md., 2005 (“USP/NF”), or a more recent edition, and the components listed in the continuously updated Inactive Ingredient Search online database of the FDA. Other useful components that are not described in the USP/NF, etc. may also be used.

As used herein, the term “pharmaceutically acceptable carrier” means a non-toxic, inert solid, semi-solid or liquid filler, diluent, encapsulating material, or formulation auxiliary of any type. The use of such media and agents for pharmaceutical active substances is well known in the art (see generally Remington's Pharmaceutical Sciences (A. R. Gennaro, Ed.), 21st edition (2005)). For example, a pharmaceutical composition for oral administration can be formulated using pharmaceutically acceptable carriers known in the art in dosages suitable for oral administration. Such carriers enable the pharmaceutical compositions to be formulated as tablets, pills, dragees, capsules, liquids, gels, syrups, slurries, suspensions, and the like, for ingestion by the subject.

Pharmaceutically acceptable carriers comprising excipients and auxiliaries that facilitate processing of the active compounds into preparations which can be used pharmaceutically. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil; and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen-free water; isotonic saline; Ringer's solution; ethyl alcohol; artificial cerebral spinal fluid (CSF), and phosphate buffer solutions, as well as other non-toxic compatible lubricants such as sodium lauryl sulfate and magnesium stearate, as well as coloring agents, releasing agents, coating agents, sweetening, flavoring, and perfuming agents, preservatives and antioxidants can also be present in the composition, according to the judgment of the formulator based on the desired route of administration.

The pharmaceutically acceptable also include polymers. Particularly suitable polymers are poloxamers.

The pharmaceutical composition may further include a protease. Particularly suitable proteases can be trypsin, collagenase, and combinations thereof.

The pharmaceutical composition may further include a small molecule.

Except insofar as any conventional media or agent is incompatible with a compound, its use in a pharmaceutical composition is contemplated. Supplementary active ingredients can also be incorporated into the compositions.

A “stable” formulation or composition can refer to a composition having sufficient stability to allow storage at a convenient temperature, such as between about 0° C. and about 60° C., for a commercially reasonable period of time, such as at least about one day, at least about one week, at least about one month, at least about three months, at least about six months, at least about one year, or at least about two years.

The composition should suit the mode of administration. The compounds of use with the current disclosure can be formulated by known methods for administration to a subject using several routes which include, but are not limited to, parenteral, oral, topical, intradermal, intranasal, intramuscular, intraperitoneal, intravenous, intra-arterial, subcutaneous, epidural, transdermal, buccal, and rectal. The compounds may also be administered in combination with one or more additional agents or together with other biologically active or biologically inert agents. Such biologically active or inert agents may be in fluid or mechanical communication with the agent(s) or attached to the agent(s) by ionic, covalent, Van der Waals, hydrophobic, hydrophilic or other physical forces.

Controlled-release (or sustained-release) compositions may be formulated to extend the activity of the compound(s) and reduce dosage frequency. Controlled-release compositions can also be used to effect the time of onset of action or other characteristics, such as blood levels of the compound, and consequently affect the occurrence of side effects. Controlled-release compositions may be designed to initially release an amount of a compound(s) that produces the desired therapeutic effect, and gradually and continually release other amounts of the compound to maintain the level of therapeutic effect over an extended period of time. In order to maintain a near-constant level of a compound in the body, the compound can be released from the dosage form at a rate that will replace the amount of compound being metabolized or excreted from the body. The controlled-release of a compound may be stimulated by various inducers, e.g., change in pH, change in temperature, enzymes, water, or other physiological conditions or molecules.

Compositions, rAAV vectors, or rAAV particles described herein can also be used in combination with other therapeutic modalities. Thus, in addition to the therapies described herein, one may also provide to the subject other therapies known to be efficacious for treatment of the disease, disorder, or condition.

### Methods

Further aspects of the present disclosure are directed to methods of delivering the rAAV vectors, rAAV particles, and pharmaceutical compositions described herein into cells.

A particular aspect is directed to a method of delivering nucleic acids into a cell, the method comprising administering to the cell a recombinant adeno-associated virus (rAAV) nucleic acid vector comprising inverted terminal repeats (ITRs) free of 5′-cytosine-phosphate-guanine-3′ (CpG) motifs.

Another particular aspect is directed to a method of delivering nucleic acids into a cell, the method comprising administering to the cell a recombinant adeno-associated virus (rAAV) particle comprising a viral capsid and a rAAV nucleic acid vector comprising inverted terminal repeats (ITRs) free of 5′-cytosine-phosphate-guanine-3′ (CpG) motifs.

Another particular aspect is directed to a method of delivering nucleic acids into a cell, the method comprising administering to the cell a pharmaceutical composition comprising a pharmaceutically acceptable carrier and a recombinant adeno-associated virus (rAAV) particle comprising a viral capsid and an rAAV nucleic acid vector comprising inverted terminal repeats (ITRs) free of 5′-cytosine-phosphate-guanine-3′ (CpG) motifs.

The dose of a viral construct to be administered is based on the vector genome (vg) copy number, which is a well-established unit of measurement in the AAV viral arts. Suitable dose ranges from about 1×10² vg/injection site to about 1×10¹⁵ vg/kg (in volumes ranging from about 1 microliters to about 50 milliliters) are used. Higher or lower doses may be used, depending on, for example, route of administration, the type and severity of the disease, or the age, sex, body weight, and condition in the individual. Guidance as to particular dosages and methods of delivery is provided in the literature and generally available to practitioners in the art. In general, lower doses can be administered when a parenteral route is employed. Thus, for example, for intravenous administration, a dose in the range, for example, from about 1×10⁹ vg/kg to 1×10¹⁵ vg/kg can be used.

Particularly suitable cells are mammalian cells, including cells from experimental animals such as rodents (e.g., mice and rats), pigs, primates, rabbits, cows, horses, dogs, and the like. Cells can also be cells in a living animal, such as an experimental animal, a livestock animal, or a pet.

Particularly suitable cells are human cells. Cells can be experimental cells from human origin, including diseased or disease-free cells. Cells can also be cells in a living human patient. Cells can also be embryonic stem cells or induced pluripotent stem cells.

Further aspects of the present disclosure are directed to methods of gene therapy or methods of treating a disease in a subject in need thereof, the methods comprising administering to the subject a therapeutically effective amount of the rAAV vectors, the rAAV particles, or the pharmaceutical composition described herein.

A particular aspect is directed to method of gene therapy in a subject in need thereof, the method comprising administering to the subject a therapeutically effective amount of a recombinant adeno-associated virus (rAAV) nucleic acid vector comprising inverted terminal repeats (ITRs) free of 5′-cytosine-phosphate-guanine-3′ (CpG) motifs.

Another aspect is directed to methods of gene therapy in a subject in need thereof, the method comprising administering to the subject a therapeutically effective amount of a recombinant adeno-associated virus (rAAV) particle comprising a viral capsid and a rAAV nucleic acid vector comprising inverted terminal repeats (ITRs) free of 5′-cytosine-phosphate-guanine-3′ (CpG) motifs

Another aspect is directed to methods of gene therapy in a subject in need thereof, the method comprising administering to the subject a therapeutically effective amount of a pharmaceutical composition comprising a pharmaceutically acceptable carrier and a recombinant adeno-associated virus (rAAV) particle comprising a viral capsid and an rAAV nucleic acid vector comprising inverted terminal repeats (ITRs) free of 5′-cytosine-phosphate-guanine-3′ (CpG) motifs.

A particular aspect is directed to methods of treating a disease in a subject in need thereof, the method comprising administering to the subject a therapeutically effective amount of a recombinant adeno-associated virus (rAAV) nucleic acid vector comprising inverted terminal repeats (ITRs) free of 5′-cytosine-phosphate-guanine-3′ (CpG) motifs.

Another aspect is directed to methods of treating a disease in a subject in need thereof, the method comprising administering to the subject a therapeutically effective amount of a recombinant adeno-associated virus (rAAV) particle comprising a viral capsid and a rAAV nucleic acid vector comprising inverted terminal repeats (ITRs) free of 5′-cytosine-phosphate-guanine-3′ (CpG) motifs

Another aspect is directed to methods of treating a disease in a subject in need thereof, the method comprising administering to the subject a therapeutically effective amount of a pharmaceutical composition comprising a pharmaceutically acceptable carrier and a recombinant adeno-associated virus (rAAV) particle comprising a viral capsid and an rAAV nucleic acid vector comprising inverted terminal repeats (ITRs) free of 5′-cytosine-phosphate-guanine-3′ (CpG) motifs.

The pharmaceutical composition can be administered by a route including, but not limited to, oral, intravenous, intramuscular, intra-arterial, intramedullary, intrathecal, intraventricular, transdermal, subcutaneous, intraperitoneal, intranasal, parenteral, topical, sublingual, or rectal means. For example, administration can be selected from the group consisting of oral, intranasal, intraperitoneal, intravenous, subcutaneous, intramuscular, intratumoral, rectal, topical, and transdermal.

The determination of a therapeutically effective dose for any one or more of the compounds described herein is within the capability of those skilled in the art. A therapeutically effective dose refers to the amount of active ingredient (compound) which provides the desired result. The exact dosage will be determined by the practitioner, in light of factors related to the subject that requires treatment. Dosage and administration are adjusted to provide sufficient levels of the active ingredient or to maintain the desired effect. Factors which can be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions can be administered every 3 to 4 days, every week, or once every two weeks depending on the half-life and clearance rate of the particular formulation.

Each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

As used herein, “individual in need thereof” and “subject in need thereof” refers to an individual susceptible to or at risk of or suffering from a specified disease, disorder, or condition. Individuals may be susceptible to or at elevated risk for these diseases, disorders or conditions due to family history, age, environment, and/or lifestyle. The individual in need thereof can be an adult individual, a child, and a pediatric individual. Particularly suitable individuals can be humans Other particularly suitable individuals can be experimental animals such as rodents (e.g., mice and rats), pigs, primates, rabbits, cows, horses, dogs, and the like.

In some embodiments, the individual in need thereof is selected from the group consisting of an adult individual, a child, and a pediatric individual.

In some embodiments, the disease may refer to a liver disease, heart disease, lung disease, kidney disease, blood disorder, central nerve system disease, neuromuscular disease.

A particularly suitable administration method is in situ application to a tissue or organ. For example, the disease may be a neuromuscular disease, the expression cassette may encode for micro-dystrophin, and the administering may be to a muscle tissue or intravenous injection.

Another particularly suitable administration method can be in situ application to or near an eye. For example, the disease may be a retinal disease, and the administering may be to or near an eye.

Another particularly suitable administration method can be in situ application to or in an ear. For example, the disease may be a hearing disorder or hearing loss, and the administering may be to or in an ear.

### EXAMPLES

Examples 1-4 are directed to the design and generation of example rAAV vectors with CpG-free ITRs. Examples 5-9 are directed to in vivo mouse model experiments employing the example rAAV vectors.

### Example 1

This example presents the design of example CpG-free ITRs for the example rAAV vectors.

To determine whether CpG depletion affects rAAV production, two types of rAAV micro-dystrophin vectors that carry the identical expression cassette but differ in the ITR were made. One type had the wild-type ITR and the other type had CpG-free ITRs.

Generation of the CpG-free ITR. In one example, the CpG-free ITRs were designed based on the wild-type ITRs of AAV2. The 5′-end CpG-free ITR was designed by replacing guanine in the first CpG motif in the A sequence of the ITR with thymine, replacing guanine in the remaining three CpG motifs in the A sequence of the ITR with adenine, replacing guanine in the first CpG motif and its immediate downstream cytosine in the C sequence of the ITR with adenine and guanine, replacing guanine in the second CpG motif and its immediate downstream guanine in the C sequence of the ITR with cytosine and thymine, replacing guanine in the first CpG motif in the B sequence of the ITR with adenine, and replacing guanine in the second CpG motif in the B sequence of the ITR with cytosine. Corresponding bases in the A′, B′ and C′ sequences of the 5′-end ITR were modified with complementary bases (FIG. 1C).

In another example, the 5′-end CpG-free ITR was designed by replacing guanine in the first CpG motif in the A sequence of the ITR with thymine, replacing guanine in the remaining three CpG motifs in the A sequence of the ITR with adenine, replacing guanine in the first CpG motif and its immediate downstream cytosine in the C sequence of the ITR with thymine and guanine, replacing cytosine in the second CpG motif and guanine immediate downstream of the second CpG in the C sequence of the ITR with adenine and adenine, replacing guanine in the first CpG motif in the B sequence of the ITR with thymine, and replacing cytosine in the second CpG motif in the B sequence of the ITR with guanine. Corresponding bases in the A′, B′ and C′ sequences of the 5′-end ITR were modified with complementary bases.

In one example, the 3′-end CpG-free ITR was designed by replacing guanine in the first CpG motif in the A sequence of the ITR with thymine, replacing guanine in the remaining three CpG motifs in the A sequence of the ITR with adenine, replacing guanine in the first CpG motif in the B sequence of the ITR with adenine, replacing cytosine and guanine in the second CpG motif of the B arm with guanine and cytosine, respectively, replacing guanine in the first CpG motif and its immediate downstream cytosine in the C sequence of the ITR with adenine and guanine, and replacing guanine in the second CpG motif and its immediate downstream guanine in the C sequence of the ITR with cytosine and thymine. Corresponding bases in the A′, B′ and C′ sequences of the 5′-end ITR were modified with complementary bases (FIG. 1D).

In another example, the 3′-end CpG-free ITR was designed by replacing guanine in the first CpG motif in the A sequence of the ITR with thymine, replacing guanine in the remaining three CpG motifs in the A sequence of the ITR with adenine, replacing guanine in the first CpG motif in the B sequence of the ITR with thymine, replacing cytosine in the second CpG motif of the B arm with guanine, replacing guanine in the first CpG motif and its immediate downstream cytosine in the C sequence of the ITR with thymine and guanine, and replacing cytosine in the second CpG motif and its immediate downstream guanine in the C sequence of the ITR with adenine and adenine. Corresponding bases in the A′, B′ and C′ sequences of the 5′-end ITR were modified with complementary bases.

The designed CpG-free ITRs were synthesized by GenScript (Piscataway, N.J.). The designed CpG-free ITRs can also be synthesized by any other commercial resources that provide DNA synthesis service.

FIG. 1. Engineering of the CpG-free ITR. FIG. 1A, Schematic outline of the AAV vector. The expression cassette was composed of a promoter, a transgene and a poly-adenylation (pA) signal, and other undepicted regulatory elements (such as an intron, an enhancer, a microRNA binding target etc.). In the context of this study, the transgene was a micro-dystrophin gene (μDys). In an AAV vector, the expression cassette was flanked by two ITRs. The 5′ and 3′ ITRs are highlighted by the dotted boxes. FIG. 1B, alignment of the 3′-ITR from the version-1 CpG-free vector and AAV1, 2, 3, 4, 6, and 7. The AAV ITR was divided into D, A, B, B′, C, C′ and A′ sections. Bold black letters mark the nucleotides in the AAV2 ITR that were different from these in the version 1 CpG-free ITR. The underlined italic nucleotides GTTGGCC between section D and section A are the AAV2 terminal resolution site (trs). The underlined italic nucleotides CTTTG between section C and section C′ are the AAV2 second Rep-binding element (RBE′). The underlined nucleotides in sections A and A′ are the AAV2 Rep-binding element (RBE). Boxes mark the GAGY (RCTC in the complementary strand) tetranucleotide repeat motif in the Rep-binding element. Asterisks indicate nucleotides that are conserved in all ITRs. Black dots indicate nucleotides that are conserved in the ITR of AAV1, 2, 3, 4, 6, and 7 but not in the version-1 CpG-free ITR. Dashes mark nucleotides absent in the version-1 CpG-free ITR. FIG. 1C, two-dimensional drawing of the 5′-ITR in the flop configuration. The AAV ITR was divided into four regions including the A/A′ stem (sequence A and its complimentary sequence A′), B/B′ arm (sequence B, its complimentary sequence B′ and three intervening adenine nucleotides between sequences B and B′), C/C′ arm (sequence C, its complimentary sequence C′ and three intervening thymidine nucleotides between sequences C and C′), and D-sequence (underlined). In addition, there is an unpaired thymidine between the B/B′ and C/C′ arm. Gray letters, nucleotides deleted in the AAV vector. RBE, Rep-binding element, a 22-bp sequence. The core RBE sequence (box) consisted of a 10-bp sequence. RBE′, the second Rep-binding element, a 5-base sequence. Arrowhead, terminal resolution site (trs). Insert, explanation of the terminology. Nucleotides modified in the CpG-free ITR are marked. FIG. 1D, two-dimensional drawing of the 3′-ITR in the flop configuration. The 3′-ITR is divided into four regions including the A/A′ stem (sequence A and its complimentary sequence A′), B/B′ arm (sequence B, its complimentary sequence B′ and three intervening adenine nucleotides between sequences B and B′), C/C′ arm (sequence C, its complimentary sequence C′ and three intervening thymidine nucleotides between sequences C and C′), and D-sequence (underlined). In addition, there is an unpaired adenine between the B/B′ and C/C′ arm. Gray letters, nucleotides deleted in the AAV vector. RBE, Rep-binding element, a 22-bp sequence. The core RBE sequence (box) consisted of a 10-bp sequence. RBE′, the second Rep-binding element, a 5-base sequence. Arrowhead, terminal resolution site (trs). Insert, explanation of the terminology. Nucleotides modified in the CpG-free ITR are marked. FIG. 1E, alignment of the 3′-ITR from the version-2 CpG-free vector and AAV1, 2, 3, 4, 6, and 7. The AAV ITR is divided into D, A, B, B′, C, C′ and A′ sections. Bold black letters mark the nucleotides in the AAV2 ITR that are different from these in the version 2 CpG-free ITR. Shaded letters mark the nucleotides that were different between version-1 and version-2 CpG-free ITR. The underlined italic nucleotides GTTGGCC between section D and section A were the terminal resolution site (trs). The underlined italic nucleotides CTTTG between section C and section C′ were the second Rep-binding element (RBE′). The underlined nucleotides in sections A and A′ are the AAV2 Rep-binding element (RBE). Boxes mark the GAGY (RCTC in the complementary strand) tetranucleotide repeat motif in the Rep-binding element. Asterisks indicate nucleotides that are conserved in all ITRs. Black dots indicate nucleotides that are conserved in the ITR of AAV1, 2, 3, 4, 6, and 7 but not in the version-1 CpG-free ITR. Dashes mark nucleotides absent in the version-2 CpG-free ITR.

### Example 2

This example presents production of the example rAAV vectors with CpG-free ITRs with an example expression cassette coding for micro-dystrophin.

Micro-dystrophin expression cassette. The codon-optimized human micro-dystrophin gene contained the N-terminal domain, hinge 1, spectrin-like repeats 1, 16, 17 and 24, hinge 4, the cysteine-rich domain, and the syntrophin/dystrobrevin-binding site of human dystrophin. Micro-dystrophin expression was regulated by the human elongation factor 1-α(E1F-α) promoter and the mouse cytomegalovirus enhancer from pCpGfree (Invivogen, San Diego, Calif., USA), and a synthetic polyadenylation site from pGL3-Basic (Promega, Madison, Wis., USA).

Recombinant AAV production, purification and titration. Two cis-plasmids were used for rAAV stock production. They carried exactly the same micro-dystrophin expression cassette as described above. One cis-plasmid contained the version-1 CpG-free ITRs. The other cis-plasmid contained the wild-type ITRs. The rAAV vectors were packaged in Y731F tyrosine mutant AAV-9 and vector stocks were produced using the transient transfection method according to published protocols (Shin et al., 2012; Shin et al., 2013). The rAAV vectors were purified through two rounds of isopycnic cesium chloride ultracentrifugation followed by three changes of HEPES buffer at 4° C. for 48 hr. Viral titer was determined by quantitative PCR using the Fast SYBR Green Master Mix kit (Applied Biosystems, Foster City, Calif.) in an ABI 7900 HT qPCR machine. The pair of primers were designed for the mouse cytomegalovirus enhancer region. The forward primer was 5′-ACATAAGGTCAATGGGAGGTAAGC (SEQ ID NO:13) and the reverse primer was 5′-CAATGGGACTTTCCTGTTGATTC (SEQ ID NO:14).

ITR Sequencing. Due to the complicated secondary structure and high GC content, the DNA was first amplified with the GE healthcare illustra TempliPhi Sequence Resolver Kit (GE healthcare life sciences, Code #28-9035-29). The amplified product was then subjected to Sanger sequencing using the primer 5′-GATGTGCTGCAAGGCGATTA (SEQ ID NO: 15) for the 5′-end ITR and the primer 5′-TTATGCTTCCGGCTCGTATG (SEQ ID NO:16) for the 3′-end ITR.

AAV virus ITR sequencing. Vector genomes were isolated by phenol: chloroform: isoamyl alcohol (Invitrogen) extraction and EtOH precipitation as described in Tran et al (Tran et al., 2020). Briefly, SMRT sequencing libraries were built with indexed SMRTBell adapters using the Express Template Prep Kit 2.0 (End-Repair/A-tailing) (PN 100-938-900). Libraries were run on a Sequel II with 15-hour collections. The resultant sub-reads were processed with recall adapters and ccs was run with minimum thresholds with —min−snr=2.00 and —min−passes=0.5. Reads were then demultiplexed, mapped to reference vector genomes, and displayed on IGV as described in Robinson et al (Robinson et al., 2011). AAV virus ITR sequencing confirmed the complete elimination of the CpG motifs in purified AAV vectors.

### Example 3

This example presents studies on yield of the example rAAV vectors comprising CpG-free ITRs.

Transient transfection is the most commonly used method for rAAV production and was used to make the wild-type and CpG-free vector. Crude lysate was purified side-by-side using the isopycnic cesium chloride ultracentrifugation method. The vector titer was determined by quantitative PCR using the identical setting.

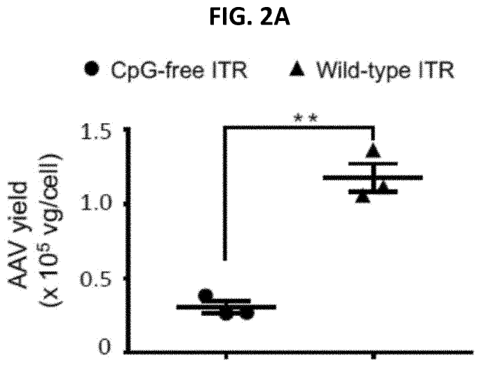

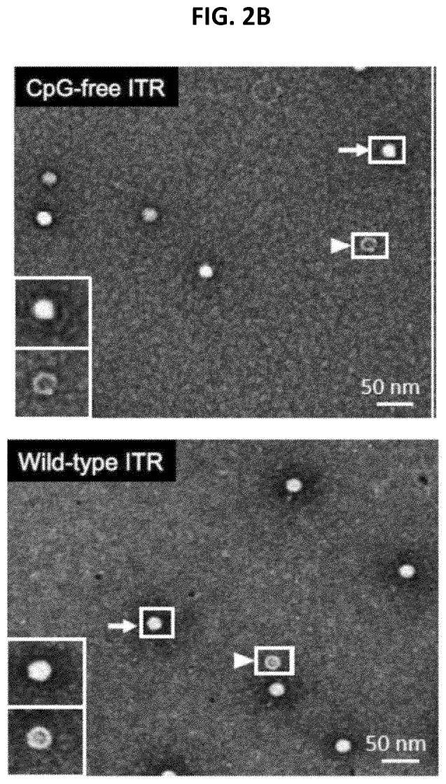

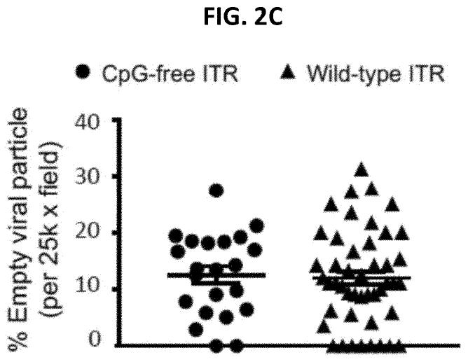

To study vector production efficiency, three batches of each vector were made. The yields from the wild-type and CpG-free vector were 1.18±0.08×10⁵ vg/cell and 3.03±0.32×10⁴ vg/cell, respectively (FIG. 2A).

FIGS. 2A-2C. Quantitative evaluation of rAAV production. FIG. 2A, Quantification of the vector yield from three independent production rounds for each vector. **, p<0.01. FIG. 2B, Representative transmission electron microscopy images of the wild-type ITR vector and CpG-free ITR vector. Arrow, a fully packaged AAV particle. Arrowhead, an empty AAV particle. FIG. 2C, Quantification of empty particles. Each data point represents the quantification result from one field at the 25,000× magnification. For the wild-type ITR vector, a total of 48 fields were quantified. For the CpG-free ITR vector, a total of 25 fields were quantified.

### Example 4

This example presents studies on genome encapsidation of the example rAAV vectors comprising CpG-free ITRs.

To study rAAV genome encapsidation, the percentage of empty particles was quantified using transmission electron microscopy (FIGS. 2B and 2C). The packaged rAAV virions showed homogenous electron density while empty particles had a dark center (FIG. 2B). On average, there were 11.9±1.2% and 11.8±1.5% of empty particles in the wild-type and CpG-free vector stock, respectively. It appeared that elimination of the CpG motif from the ITR did not influence the packaging of the single stranded genome into the pre-assembled capsid because similar amounts of empty particles were detected (˜12%) in the wild-type and CpG-free vectors (FIGS. 2B and 2C).

Electron microscopy. The rAAV particles were examined by transmission electron microscopy. Specifically, purified and dialyzed AAV virus was diluted to 1 to 3×10⁹ vg/μl with ultra-pure water and then placed on a 200-mesh glow-discharge carbon-coated copper grid for five minutes. After four to five rounds of gentle washing in ultra-pure water, virus was stained with 2% NANO-W™ (Nanoprobes, Yaphank, N.Y., USA) for 5 minutes. Viral particles were visualized using a JEOL JEM-1400 transmission electron microscope.

Examples 5-9 are directed to in vivo mouse model experiments employing the example rAAV vectors comprising CpG-free ITRs.

### Example 5

This example presents administration of the example rAAV vectors with CpG-free ITRs in a mouse model of Duchenne muscular dystrophy (DMD).

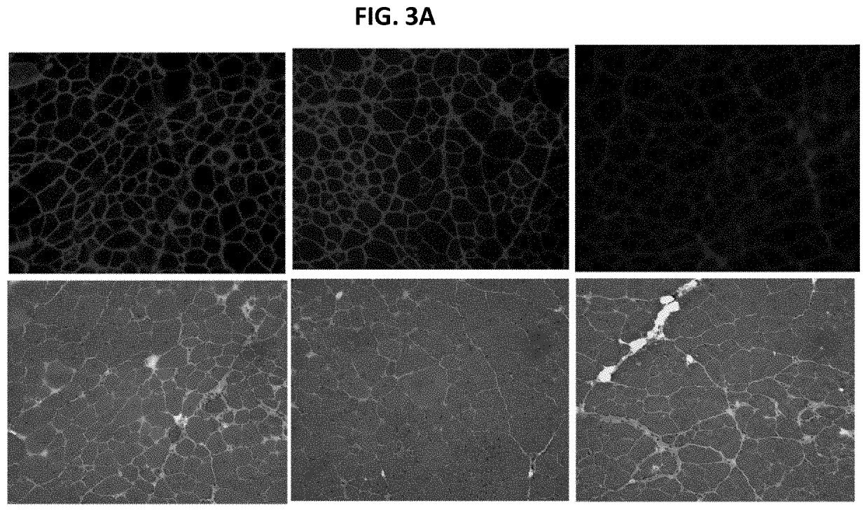

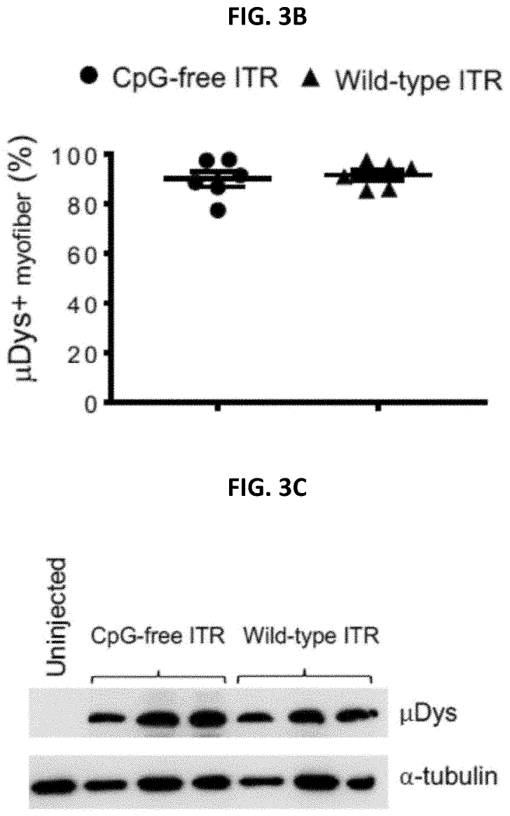

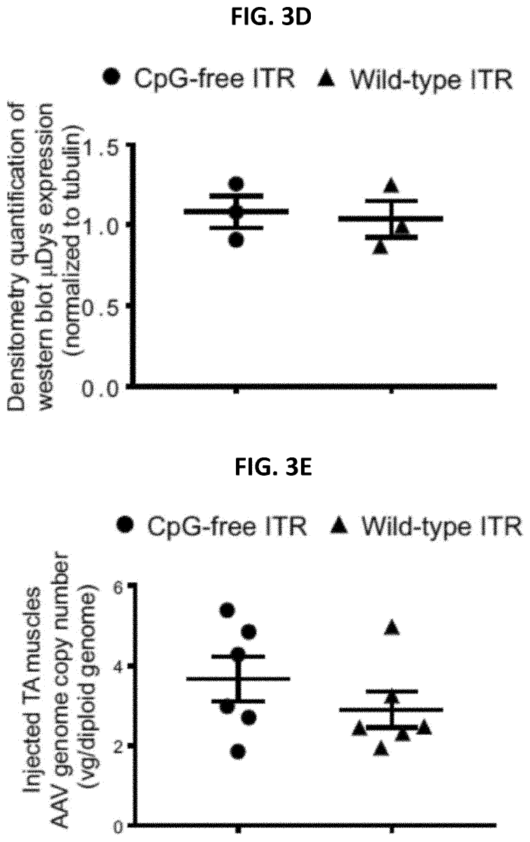

To determine whether CpG depletion affects in vivo transduction of the rAAV vector, a paired study in the mdx model of DMD was performed. Specifically, the wild-type and CpG-free vectors were injected to different sides of the tibialis anterior (TA) muscle of the same mdx mouse. Four months after rAAV injection, the micro-dystrophin expression, AAV vector genome copy number in the TA muscle, and histological and physiological rescue of the muscle disease by micro-dystrophin were quantified (FIGS. 3 to 6).

Experimental Mice. All animal experiments were approved by the Animal Care and Use Committee of the University of Missouri and were in accordance with NIH guidelines. All animal experiments were conducted at the University of Missouri. Dystrophin-deficient mdx mice (Stock number 001801) were originally purchased from The Jackson Laboratory (Bar Harbor, Me.). Experimental mice were generated in house in a specific-pathogen-free barrier facility at the University of Missouri using founders from The Jackson Laboratory. The genotype of the mice was confirmed using a published protocol (Shin et al., 2011). All mice were maintained in a specific-pathogen free animal care facility on a 12-hour light (25 lux): 12-hour dark cycle with access to PicoLab rodent diet 20 #5053 and autoclaved municipal tap water ad libitum. The room temperature and relative humidity were maintained at 68±2° F. and 50±20%, respectively. All animals were observed daily for their general condition and well-being. All mice had a unique identification number (ear tag) that was randomly assigned at the time of weaning.

rAAV administration. 2.8×10¹⁰ vg particles/muscle (in 50 μl of HEPES buffer) of the rAAV vector were injected to the TA muscle of six 10-m-old female mdx mice using a Hamilton syringe. One side the TA muscle received the wild-type vector and the contralateral side of the same mouse received the CpG-free vector.

Morphological analysis. Four months after rAAV injection, a terminal TA muscle function assay was performed. After the functional assay, mice were euthanized according to the protocols approved by the University of Missouri Animal Care and Use Committee. The TA muscle was carefully dissected out and cut into two pieces. One piece was snap-frozen in liquid nitrogen. The other piece was embedded in liquid nitrogen-cooled isopentane in the optimal cutting temperature compound (Sakura Finetek Inc., Torrance, Calif.). Ten-micron cryosections were used for staining. General muscle histopathology was revealed with haematoxylin and eosin (H&E) staining. Dystrophin expression was evaluated by immunofluorescence staining using Dys-3 (1:20, Vector Laboratories, Peterborough, UK), a species-specific dystrophin monoclonal antibody that recognizes the hinge 1 region of human dystrophin but does not cross react with mouse dystrophin. Slides were viewed at the identical exposure setting using a Nikon E800 fluorescence microscope. Images were taken with a QImage Retiga 1300 camera. Centrally nucleated myofibers were determined from digitalized H&E stained-images using the Fiji imaging software (Schindelin et al., 2012). Percentage of dystrophin positive cells was quantified from digitalized dystrophin immunostaining images using the Fiji imaging software.

Western blot. Tibialis anterior muscles were homogenized in a homogenization buffer containing 10% SDS, 5 min ethylenediaminetetraacetic acid, 62.5 min Tris-HCl (pH 6.8), and 2% protease inhibitor (Roche, Indianapolis, Ind., USA) using a tissue homogenizer (Bullet Blender Storm 24, Next Advance, NY) at the speed set 12 in the machine for 10 min at 4° C. The homogenate was centrifuged at 14,000 RPM for 3 minutes in an Eppendorf centrifuge (model 5417C; Brinkmann Instruments Inc., Westbury, N.Y.). The total protein concentration in the supernatant was measured using the DC assay kit (BioRad, Hercules, Calif.). 50 μg of protein was denatured at 95° C. for 5 min, chilled on ice for 2 min and then separated on a 3% stacking/6% separating SDS-polyacrylamide gel at 100 V. Proteins were transferred to a 0.45 μm PVDF membrane at 60 V for 10 hours at 4° C. in Towbin's buffer containing 10% methanol. The membrane was washed with distilled water for 5 min and then immersed in 10 ml 1× iBind Flex solution for at least 2 min (mixed 500 μl 100×Additive and 10 ml iBind Flex 5× buffer in 39.5 ml distilled water). The membrane was then cut into two pieces containing the micro-dystrophin and α-tubulin respectively. Then the membrane was placed with the protein-side down on the top of pooled solution on pre-wetted iBind Flex Card placed on iBind Flex Western System (Catalog number SLF 2000, Invitrogen). Samples were added into rows of the well insert in the following order: Row 1, primary antibody mouse anti-human dystrophin repeat 16 (1:200 in 1× iBind Flex solution, MANDYS102 clone 7D2 Type G2a, ex43, 2047-2105) (Morris et al., 2011) or mouse anti-α-tubulin (1:1000 in 1× iBind Flex solution, T5168, Sigma); Row 2 1× iBind Flex FD solution; Row 3, second antibody goat anti-mouse IgG (1:1000 in 1× iBind Flex solution, Santa Cruz, Dallas, Tex.); Row 4, 1× iBind Flex FD solution. The well cover was closed, and the samples were incubated for 3 hours. Signals were detected using the Clarity Western ECL substrate (BioRad, Hercules, Calif.) and visualized using the Li-COR Odyssey imaging system. Densitometry quantification of the band intensity was performed using the Li-COR Image Studio Version 5.0.21 software. The relative intensity of the micro-dystrophin protein bands was normalized to the corresponding α-tubulin band (loading control) in the same blot.

Quantification of the vector genome copy number in the TA muscle. Genomic DNA was extracted from OCT-embedded frozen tissue samples. DNA concentration was measured with NanoDrop One^{C} Spectrophotometer (ThermoFisher Scientific, Waltham, Mass., USA). Quantitative TaqMan PCR assays were performed using the PrimeTime Gene Expression Master Mix (Integrated DNA technologies IDT, IA) in an ABI 7900 HT qPCR machine (Applied Biosystems, Foster City, Calif.). The qPCR primers and probe were designed from human EF-1 α promoter region. The forward primer was 5′-GGCTTGGGTAAACTGGGAAA-3′ (SEQ ID NO:17), the reverse primer was 5 ‘-GTTCACAGAGACTACTGCACTTAT-3’ (SEQ ID NO:18) and the probe was 5′-ATGTGGTGTACTGGCTCCACCTTT-3′ (SEQ ID NO:19). The qPCR reaction was carried out under the following conditions: 10 minutes at 95° C., followed by 40 cycles: 15 seconds at 95° C. and 1 minute at 60° C. The threshold cycle (Ct) value of each reaction was converted to the vector genome copy number by measuring against the copy number standard curve of known amount of the cis-plasmid containing the version-1 CpG-free ITRs. The data was reported as the vector genome copy number per diploid genome.

Skeletal muscle function assay. The function of the TA muscle was evaluated in situ according to published protocols (Hakim et al., 2011; Hakim et al., 2013). Specifically, the twitch force, tetanic force and eccentric contraction profile were measured. Experimental mice were anesthetized via intra-peritoneal injection of a cocktail containing 25 mg/ml ketamine, 2.5 mg/ml xylazine, and 0.5 mg/ml acepromazine at 2.5 μl/g body weight. The TA muscle and the sciatic nerve were carefully exposed. The mouse was then transferred to a custom-designed thermo-controlled footplate platform (Hakim et al., 2013). Subsequently, forces were measured in situ with a 305C-LR dual-mode servomotor transducer (Aurora Scientific, Inc., Aurora, ON, Canada) according to published protocols (Hakim et al., 2011; Hakim et al., 2013). The absolute twitch force, the optimal maximal isometric tetanic force, and the force drop through 10 repetitive cycles of eccentric contraction were determined. Data acquisition and analysis were performed with the Dynamic Muscle Control and Analysis software (Aurora Scientific Inc.). The specific muscle force was calculated by dividing the absolute muscle force with the muscle cross-sectional area (CSA). Muscle CSA was calculated according to the following equation, CSA=(muscle mass, in g)/[(muscle density, in g/cm³)×(length ratio)×(optimal muscle length, in cm)]. For muscle density, 1.06 g/cm³ was used (Mendez and Keys, 1960). The length ratio refers to the ratio of the optimal fiber length to the optimal muscle length. The length ratios for the TA muscles was 0.6 (Burkholder et al., 1994).

Statistical analysis. Data are presented as mean±standard error of mean (SEM). Statistical significance was determined by the Student t-test using GraphPad PRISM software version 7.0 (GraphPad Software, La Jolla Calif.). The difference was considered significant when p<0.05.

### Example 6

This example presents the results of immunofluorescence staining of muscle tissue from the mice in the study.

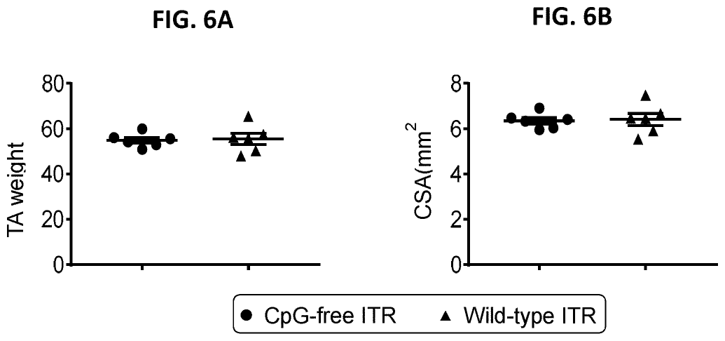

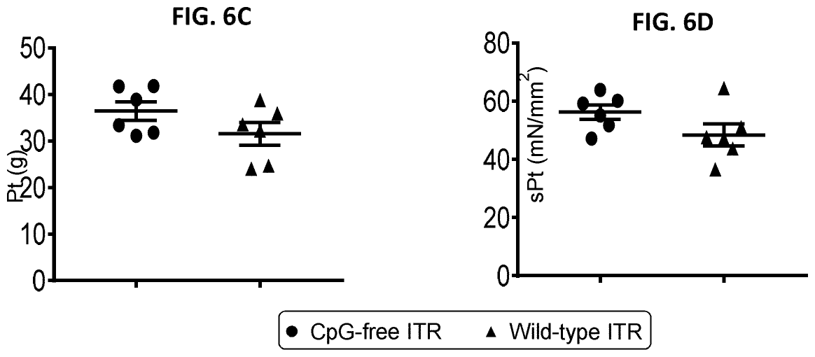

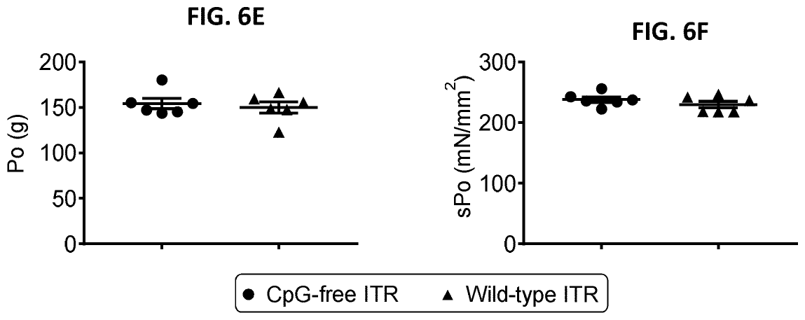

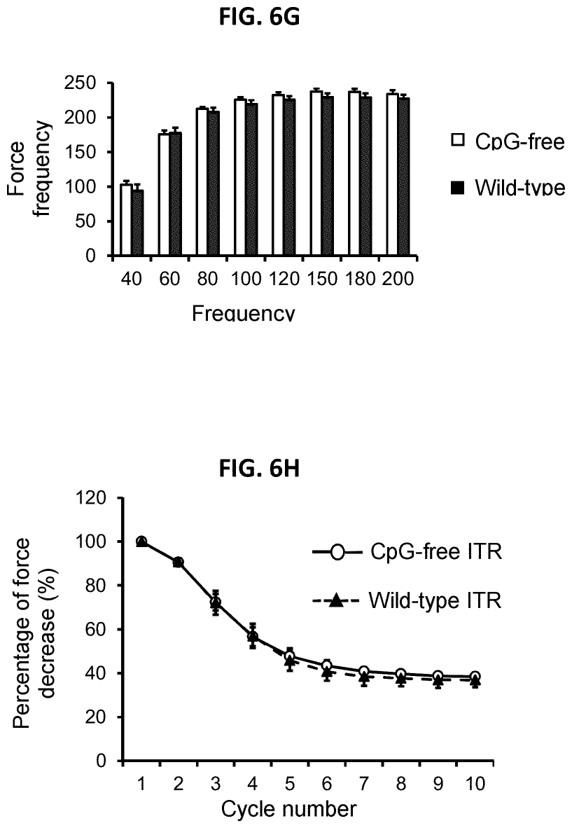

On immunofluorescence staining, robust sarcolemma micro-dystrophin expression throughout the entire muscle in both wild-type and CpG-free vector injected TA muscle was observed (FIG. 3A, FIG. 6). There was no statistical difference in the percentage of micro-dystrophin positive myofibers (FIG. 3B).

FIGS. 3A and 3B. Evaluation of micro-dystrophin expression by immunofluorescence staining. FIG. 3A, Representative dystrophin immunofluorescence staining and HE staining micrographs from the tibialis anterior muscle of dystrophin-null mdx mice that were injected with the CpG-free vector (left panels) and the wild-type vector (middle panels). The TA muscle from an age and sex-matched un-injected mdx mouse was included as the control (right panels). Scale bar applies to all images. FIG. 3B, Quantification of dystrophin positive myofibers.

FIGS. 3C and 3D. Evaluation of micro-dystrophin expression by western blot. FIG. 3C, Representative dystrophin western blot from the tibialis anterior muscle of dystrophin-null mdx mice that did not receive AAV micro-dystrophin vector injection (uninjected), injected with the CpG-free AAV micro-dystrophin vector (CpG-free ITR), and the wild-type AAV micro-dystrophin vector (wild-type ITR). FIG. 3D, Densitometry quantification of the band intensity using the Li-COR Image Studio Version 5.0.21 software. The relative intensity of the micro-dystrophin protein bands was normalized to the corresponding alpha-tubulin band (loading control) in the same blot.

FIG. 3E. Evaluation of the AAV vector genome copy number in the TA muscle by quantitative PCR. AAV vector genome copy number was quantified for the TA muscle in dystrophin-null mdx mice that either received the CpG-free AAV micro-dystrophin vector injection (CpG-free ITR) or the wild-type AAV micro-dystrophin vector injection (wild-type ITR).

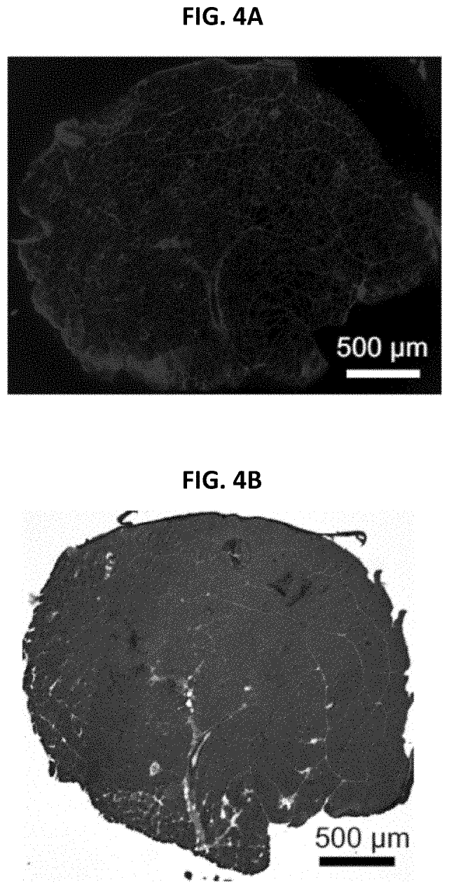

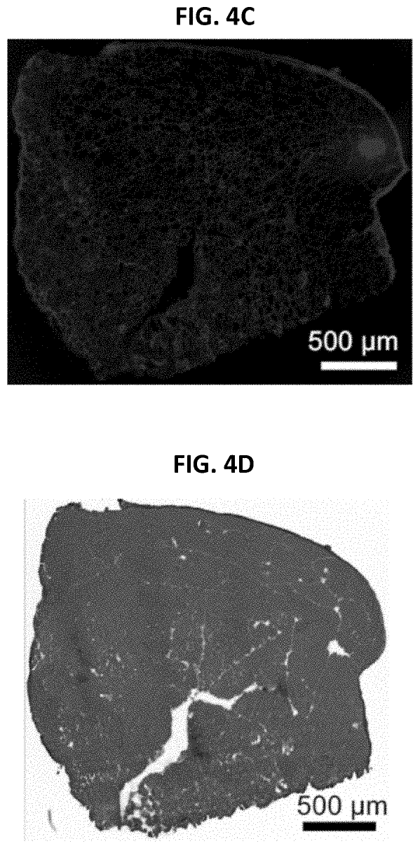

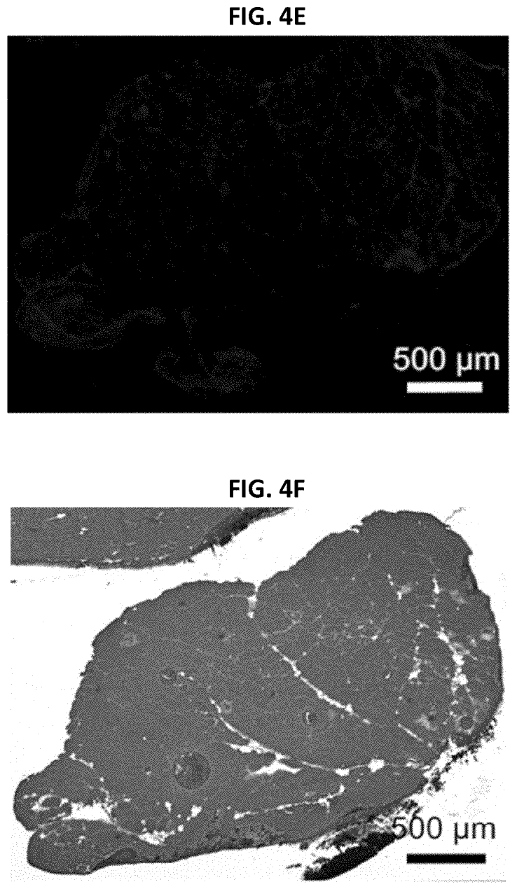

FIGS. 4A-4F. Representative full-view dystrophin immunostaining and HE staining photomicrographs of the tibialis anterior muscle. FIG. 4A, Dystrophin staining of the CpG-free vector injected muscle. FIG. 4B, HE staining of the CpG-free vector injected muscle. FIG. 4C, Dystrophin staining of the wild-type ITR vector injected muscle. FIG. 4D, HE staining of the wild-type ITR vector injected muscle. FIG. 4E, Dystrophin staining of the un-injected muscle. FIG. 4F, HE staining of the un-injected muscle

### Example 7

This example presents the results of studies on histological rescue in muscle tissue from the mice in the study.

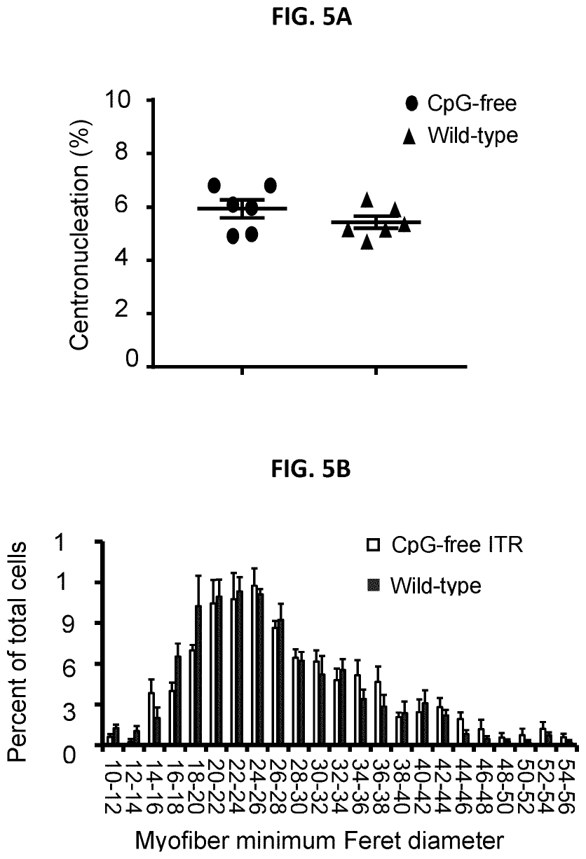

To determine histological rescue, centronucleation and myofiber size distribution was quantified (FIG. 5). The former revealed degeneration/regeneration and the later reveals muscle hypertrophy/atrophy. The wild-type vector injected muscle contained 54.3±2.1% centrally nucleated myofibers. The CpG-free vector injected muscle contained 59.1±3.1% centrally nucleated myofibers (FIG. 5A). The myofiber size was measured by the minimum Feret diameter (FIG. 5B). Throughout the entire range (from 10 to 56 μm), there was no difference between the wild-type and CpG-free vector injected muscles.

FIGS. 5A and 5B. Evaluation of centronucleation and myofiber size distribution. FIG. 5A, The percentage of myofibers that contained centrally localized nuclei in the mdx muscle treated with the CpG-free vector and the wild-type vector. FIG. 5B, The distribution of the percentage of myofibers at different minimum Feret diameters in 616 myofibers from the CpG-free vector treated muscle (n=6 muscles, 80 to 131 myofibers per muscle) and 712 myofibers from the wild-type vector treated muscle (n=6 muscles, 87 to 135 myofibers per muscle).

### Example 8

This example presents the results of studies on physiological rescue in muscle tissue from the mice in the study.

To determine physiological rescue, the muscle weight, cross-sectional area, absolute and specific twitch force, absolute and specific tetanic force, force-frequency relationship, and force drop following eccentric contraction challenge were quantified (FIG. 6). In all the parameters examined, there was no statistically significant difference.

FIGS. 6A-6H. Quantitative evaluation of muscle contractility. FIG. 6A, The weight of the tibialis anterior (TA) muscle. FIG. 6B, Muscle cross-sectional area (CSA). FIG. 6C, Absolute twitch force (Pt). FIG. 6D, Specific twitch force (sPt). FIG. 6E, Absolute tetanic force (Po). FIG. 6F, Specific tetanic force (sPo). FIG. 6G, Force-frequency relationship. FIG. 6H, Eccentric contraction profiles.

### Example 9

This example presents the results of studies on transduction efficiency in the mouse study.

Given the role the ITR plays in single strand to double strand conversion (a process akin to replication) and the observation that the yield was significantly reduced for the CpG-free vector, it was initially thought the CpG-free vector might show a reduced transduction efficiency. To test this, a paired study in the same animal was performed. The wild-type vector was injected on one side of the muscle and the CpG-free vector was injected on the contralateral side. Surprisingly, no difference in transgene expression was detected (FIG. 3). Importantly, both vectors were equally effective in attenuating histological and physiological defects in diseased mice (FIGS. 5, and 6).

In sum, a CpG-free ITR can be used to produce an rAAV vector. Importantly, the biological potency of the rAAV vector that has no CpG in the ITR was equivalent to that of the vector carrying the wild-type ITR.

When introducing elements of the present disclosure or the preferred embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the disclosure are achieved, and other advantageous results attained.

As various changes could be made in the above methods, processes, and compositions without departing from the scope of the disclosure, it is intended that all matter contained in the above description and shown in the accompanying drawings shall be interpreted as illustrative and not in a limiting sense.

### REFERENCES

Amiss, T. J., Mccarty, D. M., Skulimowski, A., and Samulski, R. J. (2003). Identification and characterization of an adeno-associated virus integration site in CV-1 cells from the African green monkey. J Virol 77, 1904-1915.
Ashley, S. N., Somanathan, S., Giles, A. R., and Wilson, J. M. (2019). TLR9 signaling mediates adaptive immunity following systemic AAV gene therapy. Cell Immunol 346, 103997.
Brister, J. R., and Muzyczka, N. (1999). Rep-mediated nicking of the adeno-associated virus origin requires two biochemical activities, DNA helicase activity and transesterification. J Virol 73, 9325-9336.
Brister, J. R., and Muzyczka, N. (2000). Mechanism of Rep-mediated adeno-associated virus origin nicking. J Virol 74, 7762-7771.
Burkholder, T. J., Fingado, B., Baron, S., and Lieber, R. L. (1994). Relationship between muscle fiber types and sizes and muscle architectural properties in the mouse hindlimb. J Morphol 221, 177-190.
Hakim, C. H., Li, D., and Duan, D. (2011). Monitoring murine skeletal muscle function for muscle gene therapy. Methods Mol Biol 709, 75-89.
Hakim, C. H., Wasala, N. B., and Duan, D. (2013). Evaluation of muscle function of the extensor digitorum longus muscle ex vivo and tibialis anterior muscle in situ in mice. J Vis Exp, e50183.
King, J. A., Dubielzig, R., Grimm, D., and Kleinschmidt, J. A. (2001). DNA helicase-mediated packaging of adeno-associated virus type 2 genomes into preformed capsids. Embo J 20, 3282-3291.
Ling, C., Wang, Y., Lu, Y., Wang, L. N., Jayandharan, G. R., Aslanidi, G. V., Li, B. Z., Cheng, B. B., Ma, W. Q., Lentz, T., Ling, C. Q., Xiao, X., Samulski, J., Muzyczka, N., and Srivastava, A. (2015) Enhanced Transgene Expression from Recombinant Single-Stranded D-Sequence-Substituted Adeno-Associated Virus Vectors in Human Cell Lines In Vitro and in Murine Hepatocytes In Vivo. Journal of Virology 89, 952-961.
Martino, A. T., Suzuki, M., Markusic, D. M., Zolotukhin, I., Ryals, R. C., Moghimi, B., Ertl, H. C., Muruve, D. A., Lee, B., and Herzog, R. W. (2011). The genome of self-complementary adeno-associated viral vectors increases Toll-like receptor 9-dependent innate immune responses in the liver. Blood 117, 6459-6468.
Mccarty, D. M. (2008). Self-complementary AAV vectors; advances and applications. Mol Ther 16, 1648-1656.
Mccarty, D. M., Fu, H., Monahan, P. E., Toulson, C. E., Naik, P., and Samulski, R. J. (2003).
Adeno-associated virus terminal repeat (TR) mutant generates self-complementary vectors to overcome the rate-limiting step to transduction in vivo. Gene Ther 10, 2112-2118.
Mendez, J., and Keys, A. (1960). Density and composition of mammalian muscle. Metabolism 9, 184-188.
Morris, G. E., Man, N. T., and Sewry, C. A. (2011). Monitoring Duchenne muscular dystrophy gene therapy with epitope-specific monoclonal antibodies. Methods Mol Biol 709, 39-61.
Needleman, S. B., and Wunsch, C. D. (1970). A general method applicable to the search for similarities in the amino acid sequence of two proteins. J Mol Biol 48, 443-453.
Pearson, W. R., and Lipman, D. J. (1988). Improved tools for biological sequence comparison. Proc Natl Acad Sci USA 85, 2444-2448.
Robinson, J. T., Thorvaldsdottir, H., Winckler, W., Guttman, M., Lander, E. S., Getz, G., and Mesirov, J. P. (2011). Integrative genomics viewer. Nature Biotechnology 29, 24-26.
Rogers, G. L., Shirley, J. L., Zolotukhin, I., Kumar, S. R. P., Sherman, A., Perrin, G. Q., Hoffman, B. E., Srivastava, A., Basner-Tschakarjan, E., Wallet, M. A., Terhorst, C., Biswas, M., and Herzog, R. W. (2017). Plasmacytoid and conventional dendritic cells cooperate in crosspriming AAV capsid-specific CD8(+) T cells. Blood 129, 3184-3195.
Rogers, G. L., Suzuki, M., Zolotukhin, I., Markusic, D. M., Morel, L. M., Lee, B., Ertl, H. C., and Herzog, R. W. (2015). Unique Roles of TLR9- and MyD88-Dependent and -Independent Pathways in Adaptive Immune Responses to AAV-Mediated Gene Transfer. J Innate Immun 7, 302-314.
Ryan, J. H., Zolotukhin, S., and Muzyczka, N. (1996). Sequence requirements for binding of Rep68 to the adeno-associated virus terminal repeats. J Virol 70, 1542-1553.
Samulski, R. J., Chang, L. S., and Shenk, T. (1987). A recombinant plasmid from which an infectious adeno-associated virus genome can be excised in vitro and its use to study viral replication. J Virol 61, 3096-3101.
Savy, A., Dickx, Y., Nauwynck, L., Bonnin, D., Merten, O. W., and Galibert, L. (2017). Impact of Inverted Terminal Repeat Integrity on rAAV8 Production Using the Baculovirus/Sf9 Cells System. Hum Gene Ther Methods 28, 277-289.
Schindelin, J., Arganda-Carreras, I., Frise, E., Kaynig, V., Longair, M., Pietzsch, T., Preibisch, S., Rueden, C., Saalfeld, S., Schmid, B., Tinevez, J. Y., White, D. J., Hartenstein, V., Eliceiri, K., Tomancak, P., and Cardona, A. (2012). Fiji: an open-source platform for biological-image analysis. Nat Methods 9, 676-682.
Shin, J.-H., Hakim, C., Zhang, K., and Duan, D. (2011). Genotyping mdx, mdx3cv and mdx4cv mice by primer competition PCR. Muscle Nerve 43, 283-286.
Shin, J.-H., Pan, X., Hakim, C. H., Yang, H. T., Yue, Y., Zhang, K., Terjung, R. L., and Duan, D. (2013). Microdystrophin ameliorates muscular dystrophy in the canine model of Duchenne muscular dystrophy. Mol Ther 21, 750-757.
Shin, J.-H., Yue, Y., and Duan, D. (2012). Recombinant adeno-associated viral vector production and purification. Methods Mol Biol 798, 267-284.
Smith, T. F., and Waterman, M. S. (1981). Identification of common molecular subsequences. J Mol Biol 147, 195-197.
Tai, P. W. L., Xie, J., Fong, K., Seetin, M., Heiner, C., Su, Q., Weiand, M., Wilmot, D., Zapp, M. L., and Gao, G. (2018). Adeno-associated Virus Genome Population Sequencing Achieves Full Vector Genome Resolution and Reveals Human-Vector Chimeras. Mol Ther Methods Clin Dev 9, 130-141.
Toth, R., Meszaros, I., Huser, D., Forro, B., Marton, S., Olasz, F., Banyai, K., Heilbronn, R., and Zadori, Z. (2019). Methylation Status of the Adeno-Associated Virus Type 2 (AAV2). Viruses 11.
Tran, N. T., Heiner, C., Weber, K., Weiand, M., Wilmot, D., Xie, J., Wang, D., Brown, A., Manokaran, S., Su, Q., Zapp, M. L., Gao, G. P., and Tai, P. W. L. (2020). AAV-Genome Population Sequencing of Vectors Packaging CRISPR Components Reveals Design-Influenced Heterogeneity. Molecular Therapy-Methods & Clinical Development 18, 639-651.
Wang, X. S., Khuntirat, B., Qing, K., Ponnazhagan, S., Kube, D. M., Zhou, S., Dwarki, V. J., and Srivastava, A. (1998). Characterization of wild-type adeno-associated virus type 2-like particles generated during recombinant viral vector production and strategies for their elimination. J Virol 72, 5472-5480.
Wang, X. S., Ponnazhagan, S., and Srivastava, A. (1996). Rescue and replication of adeno-associated virus type 2 as well as vector DNA sequences from recombinant plasmids containing deletions in the viral inverted terminal repeats: selective encapsidation of viral genomes in progeny virions. J Virol 70, 1668-1677.
Wilmott, P., Lisowski, L., Alexander, I. E., and Logan, G. J. (2019). A User's Guide to the Inverted Terminal Repeats of Adeno-Associated Virus. Hum Gene Ther Methods 30, 206-213.
Zhong, L., Zhou, X., Li, Y., Qing, K., Xiao, X., Samulski, R. J., and Srivastava, A. (2008). Single-polarity Recombinant Adeno-associated Virus 2 Vector-mediated Transgene Expression In Vitro and In Vivo: Mechanism of Transduction. Mol Ther 16, 290-295.
Zhou, Q. Z., Tian, W. H., Liu, C. G., Lian, Z. H., Dong, X. Y., and Wu, X. B. (2017). Deletion of the B-B′ and C-C′ regions of inverted terminal repeats reduces rAAV productivity but increases transgene expression. Scientific Reports 7.
Zhou, X., Zeng, X., Fan, Z., Li, C., Mccown, T., Samulski, R. J., and Xiao, X. (2008). Adeno-associated virus of a single-polarity DNA genome is capable of transduction in vivo. Mol Ther 16, 494-499.
Zhu, J., Huang, X., and Yang, Y. (2009). The TLR9-MyD88 pathway is critical for adaptive immune responses to adeno-associated virus gene therapy vectors in mice. J Clin Invest 119, 2388-2398.

What is claimed is:

1. An inverted terminal repeat (ITR) free of 5′-cytosine-phosphate-guanine-3′ (CpG) motifs.

2. The ITR of claim 1, comprising about 70% to about 90% sequence identity to at least one of SEQ ID NO:7, SEQ ID NO:9, SEQ ID NO:11, and SEQ ID NO:12.

3. The ITR of claim 1, comprising about 100 to about 200 nucleic acids.

4. The ITR of claim 1, comprising a GC content less than 70%.

5. The ITR of claim 1, comprising a Rep binding element (RBE) comprising a transition mutation.

6. The ITR of claim 1, wherein the ITR is a 5′ end ITR or a 3′ end ITR.

7. The ITR of claim 1, wherein the ITR is a 5′ end ITR and the 5′-end ITR comprises a guanine to thymine substitution in a first CpG motif in an A segment of the 5′-end ITR.

8. The ITR of claim 7, wherein the ITR is a 5′ end ITR and the 5′-end ITR comprises a guanine to adenine substitution in three remaining CpG motifs in the A segment.

9. The ITR of claim 8, wherein corresponding bases in A′, B′ and C′ segments of the 5′-end ITR are substituted with complementary bases.

10. The ITR of claim 1, wherein the ITR is a 3′ end ITR and the 3′-end ITR comprises a guanine to thymine substitution in a first CpG motif in an A segment of the 3′-end ITR.

11. The ITR of claim 10, wherein the ITR is a 3′ end ITR and the 3′-end ITR comprises a guanine to adenine substitution in three remaining CpG motifs in the A segment of the 3′-end ITR.

12. The ITR of claim 11, wherein corresponding bases in the A′, B′ and C′ segments of the 3′-end ITR are substituted with complementary bases.

13. The ITR of claim 1, comprising about 80% sequence identity to at least one of SEQ ID NO:7, SEQ ID NO:9, SEQ ID NO:11, and SEQ ID NO:12.

14. The ITR claim 1 selected from the group consisting of at least one of SEQ ID NO:7, SEQ ID NO:9, SEQ ID NO:11, and SEQ ID NO:12.

15. A recombinant adeno-associated virus (rAAV) nucleic acid vector comprising inverted terminal repeats (ITRs) free of 5′-cytosine-phosphate-guanine-3′ (CpG) motifs.

16. A recombinant adeno-associated virus (rAAV) particle comprising a viral capsid and a rAAV nucleic acid vector comprising inverted terminal repeats (ITRs) free of 5′-cytosine-phosphate-guanine-3′ (CpG) motifs.

17. A pharmaceutical composition comprising a pharmaceutically acceptable carrier and the recombinant adeno-associated virus (rAAV) particle of claim 16.

18. A method of delivering a nucleic acid into a cell, the method comprising administering to the cell the rAAV vector of claim 15.

19. A method of preventing or treating a disease in a subject in need thereof, the method comprising administering to the subject a therapeutically effective amount of the rAAV vector of claim 15.

20. The method of preventing or treating disease of claim 19, wherein the disease is selected from neuromuscular diseases, retinal diseases, hearing diseases, liver diseases, kidney diseases, lung diseases, heart diseases, blood disorders, central nerve system diseases, and other diseases.
